(19) United States
(12) Patent Application Publication
(10) Pub. No.: US 2025/0381139 A1
(43) Pub. Date: Dec. 18, 2025

(54) MULTI-TAIL TYPE IONIZABLE LIPID, PREPARATION METHOD THEREFOR AND USE THEREOF

(71) Applicant: South China University of Technology, Guangzhou (CN)

(72) Inventors: Yuan Zhang, Guangzhou (CN); Zhengqiang FU, Guangzhou (CN); Fei Gu, Guangzhou (CN)

(73) Assignee: South China University of Technology, Guangzhou (CN)

(21) Appl. No.: 19/317,038

(22) Filed: Sep. 2, 2025

Related U.S. Application Data
(63) Continuation of application No. PCT/CN2023/128610, filed on Oct. 31, 2023.

(30) Foreign Application Priority Data
Apr. 7, 2023 (CN) 202310373284.8

(51) Int. Cl. A61K 9/1271; A61K 9/1277; A61K 9/51; A61K 45/06; C07C 269/06; C07C 271/22; C07C 271/34; C07D 207/06; C07D 207/09; C07D 211/14; C07D 295/13

(52) U.S. Cl. CPC A61K 9/1271; A61K 9/1277; A61K 9/5123; A61K 9/5192; A61K 45/06; C07C 269/06; C07C 271/22; C07C 271/34; C07D 207/06; C07D 207/09; C07D 211/14; C07D 295/13; C07C 2602/48

(57) ABSTRACT

A multi-tail type ionizable lipid, a preparation method therefor and the use thereof are disclosed. The structural formula of the multi-tail type ionizable lipid of the present invention is as follows,

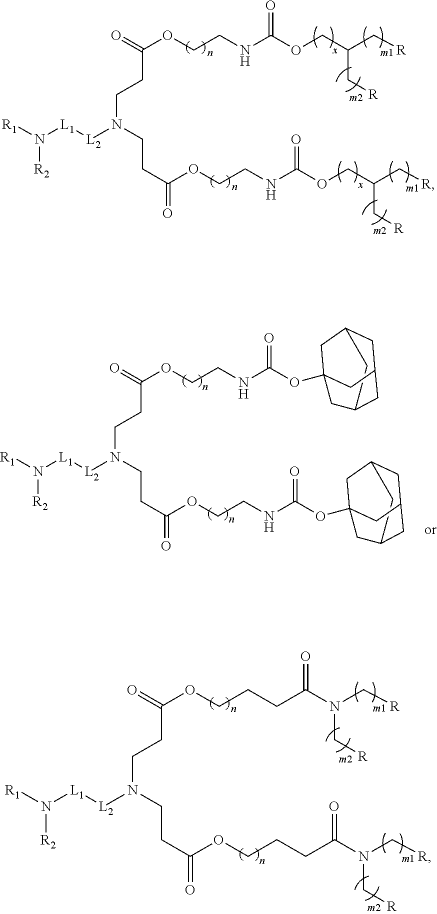

wherein R₁ and R₂ are the same or different, and each is hydrogen or an alkyl chain or an alkyl ring consisting of 1 to 6 carbons, or R₁ and R₂ together form a nitrogen-containing alkyl ring; L₁ and L₂ are the same or different, and each is an alkyl chain or an unsaturated hydrocarbyl group consisting of 1 to 6 carbons in length; and R is an alkyl group, an alkyl ring, an unsaturated hydrocarbyl group, or a heterohydrocarbyl group; and n=1 to 6, m1=1 to 15, m2=1 to 15, and x=0 to 5.

### CROSS-REFERENCE TO RELATED APPLICATIONS

This application is a continuation of International Application No. PCT/CN2023/128610, filed on Oct. 31, 2023, which claims priority to Chinese Patent Application No. 202310373284.8, filed on Apr. 7, 2023. The disclosure of the aforementioned applications are hereby incorporated by reference in their entireties.

### TECHNICAL FIELD

The present disclosure belongs to the technical field of drug carrier, specifically related to a multi-tail type ionizable lipid, preparation method therefor and use thereof.

### BACKGROUND ART

Ribonucleic acid (RNA) therapy mainly includes antisense oligonucleotides (ASOs), small interfering RNA (siRNA), small molecule RNA (miRNA) messenger RNA (mRNA), circular RNA (circRNA), which have shown great potential in treating a wide range of diseases by manipulating different modes of action. However, due to the inherent negative charge and instability of RNA molecules, it is difficult for RNA to break through biological barriers and reach the cytoplasm. To overcome this issue, RNA requires a safe, effective, and stable delivery system to protect the nucleic acid from degradation and accelerate cellular uptake and effective release of RNA. Lipid nanoparticles (LNPs) have successfully entered clinical research as a delivery system. Specifically, LNPs-mRNA vaccine has been used for the clinical treatment of Corona Virus Disease 2019 (COVID-19). This is an important milestone for LNP delivery systems. In addition, as a potential nucleic acid drug, DNA also requires an efficient delivery system. Currently, some DNA vaccines have been approved as veterinary drugs, such as West Nile Virus for horses and canine melanoma.

There are generally four types of cancer vaccines, including tumor or immune cell vaccines, polypeptide vaccines, viral carrier vaccines, and nucleic acid vaccines. Nucleic acid-based vaccines are a promising type of vaccine (DNA or RNA vaccine). Firstly, nucleic acid vaccines can simultaneously deliver multiple antigens such as tumor-associated antigens (TAAs) or somatic tumor mutations, triggering humoral immunity and cellular immunity and reducing vaccine resistance. Secondly, unlike polypeptide vaccines, nucleic acid vaccines allow Antigen-presenting cells (APCs) to simultaneously or cross present multiple epitopes of Class I and Class II patient specific human leukocyte antigens (HLA), thus being less restricted by human HLA types and more likely to stimulate a wider range of T-cell reaction. Finally, the nucleic acid vaccine is non-infectious and will not be contaminated by protein or viral sources during production. Therefore the nucleic acid vaccine is considered to have good tolerability in preventive and therapeutic applications. Meanwhile, lipid nanoparticles are also one of the key supporting carriers for cancer immunotherapy, playing an important role in national economy and people's livelihood fields such as infectious disease vaccine, cancer vaccine, and small molecule drug delivery. Therefore, in-depth research on lipid delivery carrier has both important scientific significance and promising application prospects.

In various different nucleic acid delivery systems, two categories of viral carrier and non-viral carrier can be roughly divided. Among them, the transfection efficiency of the viral carrier is relatively high, but there are issues such as safety and poor targeting. Over the past few decades, liposomes have developed rapidly as a representative non-viral carrier, and a new type of lipid-ionizable lipid—has been developed, which can be protonated at weak acid pH. The ionizable lipid is made to have positive charges, but it still remains neutral at physiological pH. The pH sensitivity of ionizable lipid is beneficial for the in vivo delivery of mRNA, as neutral lipid has less interaction with blood cell anion membranes, thereby improving the biocompatibility of nanoparticles. When lipid nanoparticles are in a weak acidic pH endosome, the ionizable lipid gains charges to promote membrane instability and increase the escape of nanoparticles from the endosome. Compared to traditional cationic liposome, the ionizable lipid has greatly improved stability and transfection efficiency in vivo, and exhibit electrical neutrality and low biological toxicity during transport. The present disclosure attempts to synthesize a new type of safe and efficient ionizable lipid to solve the problems in nucleic acid delivery mentioned above.

The inherent negative charge and instability of RNA molecules make it difficult for them to penetrate cells. In order to deliver RNA molecules to target cells, a safe, effective, and stable delivery system is required for RNA molecules to protect the nucleic acid from degradation and ensure the effective release of RNA molecules. In different types of delivery systems, lipid nanoparticles have been widely studied due to their unique properties such as simple chemical synthesis of lipids, scalability of LNP processes, and strong encapsulation capacity. However, traditional nucleic acid delivery system suffers from low efficiency, high toxicity, and poor targeting (Y. Zhang, C. Sun, C. Wang, K. E. Jankovic, Y. Dong, Lipids and Lipid Derivatives for RNA Delivery, Chem, Rev., 2021, 121, 12181-12277). The purpose of the present disclosure is to provide a preparation method and application of multi-tail type ionizable lipid. This lipid nanoparticle may efficiently deliver mRNA, circRNA, pDNA, and siRNA in mammalian cells, specifically silencing targeted gene expression. When lipid carrier reaches the intracellular environment through endocytosis, how to achieve rapid escape from the endosome is a major challenge that efficient delivery system needs to solve firstly. The multi-tail type ionizable lipid of the present disclosure generally has 2-4 more tail groups in structure than double-tail lipids. Due to the increased cross-section of the tail region, this multi-tail type ionizable lipid will produce more conical structures, making it more capable of destroying nuclear bodies and enhancing delivery efficiency. The synthesis strategy of multi-tail type ionizable lipid is that: a lipid library containing numerous lipid compounds can be rapidly synthesized through orthogonal reaction rapid synthesis, and the delivery efficiency can be determined through high-throughput cell screening.

### SUMMARY

In view of the shortcomings of the existing technology, the purpose of the present disclosure is to provide a multi-tail type ionizable lipid, preparation method therefor and use thereof. The multi-tail type ionizable lipid is an ionizable lipid. The head group of this ionizable lipid is a tertiary amino group or a secondary amino group, which can obtain a proton at acidic pH and carry a positive charge. It can bind with negatively charged nucleic acid molecules or small molecule drugs through electrostatic interactions, and then self-assembling with auxiliary lipids to form a lipid nanoparticle, thereby delivering gene drugs. Based on a series of problems encountered in current gene drug delivery, such as low efficiency and high toxicity, this multi-tail type ionizable lipid balances degradability and ensures lipid safety while maintaining overall delivery efficiency in its chemical structure design. The chemical structure of this multi-tail type ionizable lipid consists of three components: (i) ionizable head group, (ii) linking group, and (iii) hydrophobic tail. Unlike the rigorous and complex synthesis route of traditional cationic lipid, the chemical skeleton of the multi-tail type ionizable lipid provided by the present disclosure is simple, the synthesis route is simple, and the reaction mechanism is clear. By Michael addition, an ionizable lipid library may be obtained, which facilitates high-throughput screening.

The objective of the present disclosure is achieved through the following technical solution.

The structural formula of the multi-tail type ionizable lipid is shown as follows:

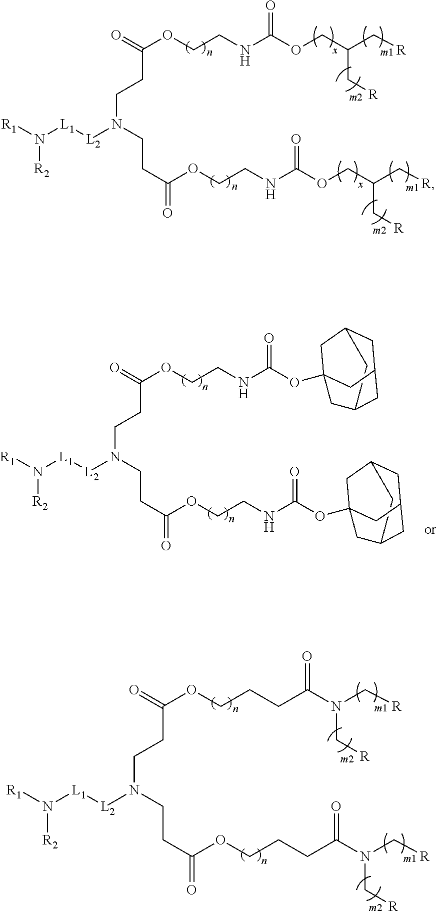

where R₁ and R₂ are same or different, each consisting of hydrogen, or an alkyl chain or an alkyl ring consisting of 1˜6 carbons, or R₁ and R₂ together form a nitrogen-containing alkyl ring; L₁ and L₂ are same or different, each consisting of an alkyl chain or an unsaturated hydrocarbon group with a length of 1˜6 carbons; R is an alkyl, an alkyl ring, an unsaturated hydrocarbon group or a heteroalkyl group; n=1˜6; m1=1˜15, m2=1˜15; and x=0˜5.

In some embodiments, the structural formula of the multi-tail type ionizable lipid includes the structural formula listed in the embodiments and following structural formula:

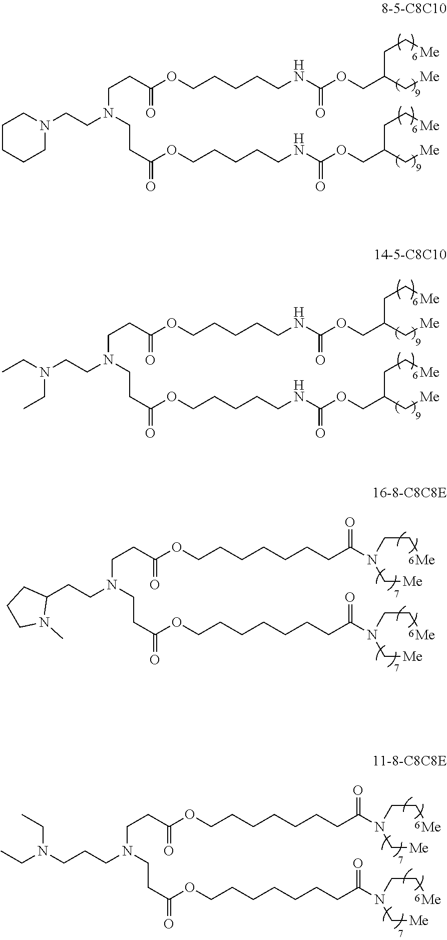

In the preparation method of the above-mentioned multi-tail type ionizable lipid, the multi-tail type ionizable lipid is obtained by performing Michael addition reaction on an organic amine compound and a tail compound containing branched chains.

The structure of the tail compound containing branched chains is as follows:

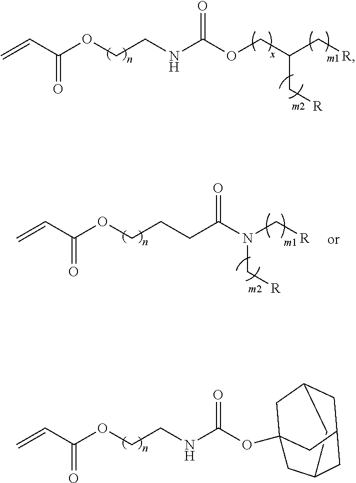

where R is an alkyl, an alkyl ring, an unsaturated hydrocarbon group or a heteroalkyl group; n=1˜6; m1=1˜15, m2=1˜15; and x=0˜5.

The organic amine compound contains at least one amino group.

In some embodiments, the organic amine compound is one of the following compounds:

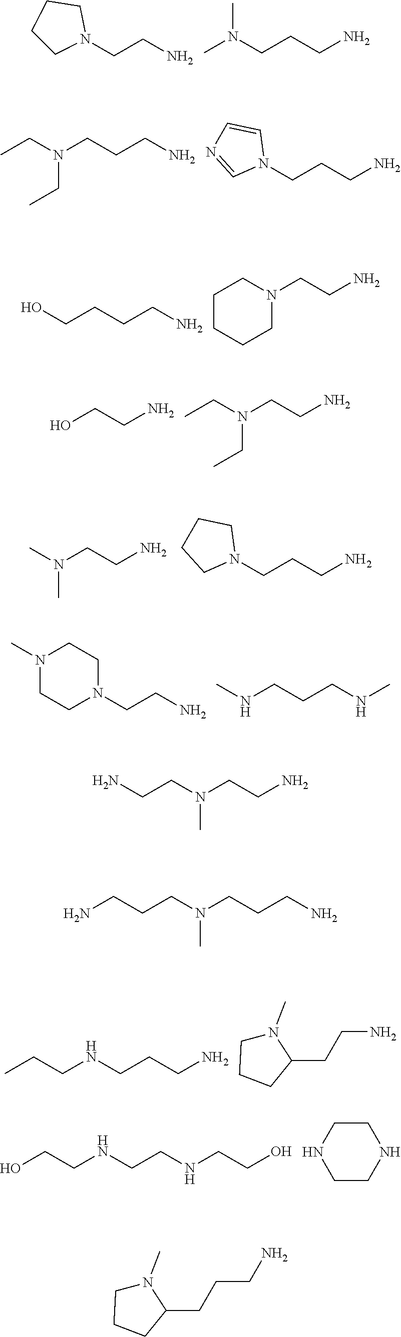

In some embodiments, the tail compound containing branched chains is obtained by esterification of acrylic acid chloride with compound 1.

The structure of compound 1 is as follows:

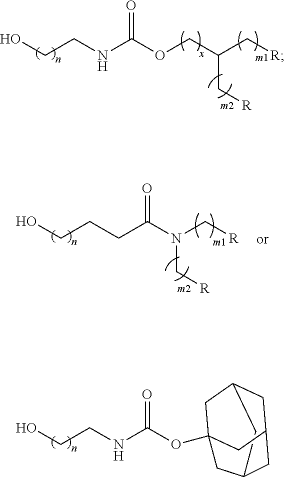

where R is an alkyl, an alkyl ring, an unsaturated hydrocarbon group or a heteroalkyl group; n=1˜6; m1=1˜15, m2=1˜15; and x=0˜5.

Application of the above-mentioned multi-tail type ionizable lipid in the preparation of drug carriers.

In some embodiments, active ingredients of the drug include nucleic acid molecule and protein drug.

Further, in some embodiments, the nucleic acid molecule includes siRNA, miRNA, circRNA, antisense RNA, CRISPR guide RNAs, replicable RNA, cyclic dinucleotides, poly IC, CpG ODN, plasmid DNA, and micro circular DNA; the protein-type drug includes cell colony-stimulating factor, interleukin, lymphotoxin, interferon protein, tumor necrosis factor, antibody, and protein antigen.

In some embodiments, the preparation method of the drug carrier includes following steps:
(a) mixing the multi-tail type ionizable lipid with cholesterol or cholesterol derivatives (such as B-sitosterol), auxiliary lipid, and an ethanol solution with polyethylene glycol modified lipid to prepare a lipid mixture solution; mixing the drug with an acidic buffer, and then mixing it well with the lipid mixture solution; incubating at a room temperature for 15 min˜1 hour, performing PBS dilution or dialysis to obtain the drug carrier;
or (b) dissolving the multi-tail type ionizable lipid with cholesterol or cholesterol derivatives in chloroform, drying with nitrogen to evaporate the solvent, adding acidic or neutral buffer and sonicating for 1˜20 minutes to prepare the liposome nanoparticles for later use; mixing fish sperm protein with the drug, then mixing with liposome nanoparticles, letting it stand for 5˜30 minutes, adding the polyethylene glycol modified lipid, and letting it stand at 30˜65° C. for 5˜20 minutes to obtain the drug carrier.

Further, in some embodiments, in step (a), a ratio of the amount of substance among the multi-tail type ionizable lipid, the cholesterol or cholesterol derivative, the auxiliary lipid, and the polyethylene glycol modified lipid is 10˜100; 0˜90; 0˜90; 0˜90; the nitrogen to phosphorus ratio of the protonated amino group and the nucleic acid drug in the multi-tail type ionizable lipid is 1˜100:1.

In step (a), auxiliary lipid includes at least one of egg yolk phospholipids, hydrogenated egg yolk phospholipids, soy phospholipids, hydrogenated soy phospholipids, sphingomyelin, phosphatidylethanolamine, bismyristoyl phosphatidylcholine, bismyristoyl phosphatidylglycerol, dipalmitoyl phosphatidylcholine, distearoyl phosphatidylcholine, dioleoyl phosphatidylethanolamine, dioleoyl phospholipids, dioleoyl phosphatidylcholine, and dipalmitoyl phosphatidylcholine.

Further, in some embodiments, in step (b), a ratio of amount of substance of the multi-tail type ionizable lipid to cholesterol or cholesterol derivatives is 1:5˜5:1; a mass ratio of the multi-tail type ionizable lipid to the drug is 1˜100:1.

Further, in some embodiments, in steps (a) and (b), the polyethylene glycol modified lipids include at least one of DSPE-PEG, C14-PEG, DMG-PEG, ALC-0159, DSPE-PEG-Maleimide, DSPE-PEG-COOH, DSPE-PEG-NH2 and chemical modified products.

Further, in some embodiments, in step (a), the acidic buffer has a pH of 3˜7; and the acidic buffer is a sodium acetate buffer or a sodium citrate buffer.

Further, in some embodiments, in step (b), the acidic buffer or the neutral buffer has a pH of 3˜7; the acidic buffer or the neutral buffer is a sodium citrate buffer, a sodium acetate buffer, or a DEPC water.

Compared with the prior art, beneficial effects of the present disclosure are as follows.
(1) For the multi-tail type ionizable lipid prepared by the present disclosure, the synthesizing method is simple, raw materials are common and readily available, and the route design is reasonable. Ionizable lipid can be prepared in large quantities through several addition reactions, facilitating high-throughput screening of materials. The obtained ionizable lipid can effectively express RNA in vitro and in vivo, and have advantages such as high efficiency and low toxicity.
(2) The chemical skeleton of the multi-tail type ionizable lipid prepared by the present disclosure contains a large number of lipid bonds, which can be quickly hydrolyzed by enzymes after effective release of RNA in vivo, making it easy to metabolize and clear in vivo, and has biodegradability. The tail structure contains branched alkyl groups, which can increase the cross-sectional area of tail of the lipid and help RNA and other drugs escape from the endosome, thereby enhancing transfection effects. Charge of the ionizable lipid can be changed with change of the pH of the environment, becoming electrically neutral under physiological conditions, reducing the cytotoxicity caused by excessive positive charges, thereby increasing the stability of lipid nanoparticles, and helping to prolong the circulation time of loaded nucleic acid drugs and improve pharmacokinetic characteristics.

### BRIEF DESCRIPTION OF THE DRAWINGS

FIG. 1 shows a hydrogen spectrum of the target product B prepared by Embodiment 2.

FIG. 2 shows a hydrogen spectrum of the target product C prepared by Embodiment 3.

FIG. 3 shows a hydrogen spectrum of ionizable lipid 3-5-C2C6 prepared by Embodiment 4.

FIG. 4 shows a hydrogen spectrum of the target product D prepared by Embodiment 5.

FIG. 5 shows a hydrogen spectrum of the target product E prepared by Embodiment 6.

FIG. 6 shows a hydrogen spectrum of the target product F prepared by Embodiment 7.

FIG. 7 shows a hydrogen spectrum of ionizable lipid 3-5-CA prepared by Embodiment 8.

FIG. 8 shows a hydrogen spectrum of the target product H prepared by Embodiment 10.

FIG. 9 shows a hydrogen spectrum of the target product I prepared by Embodiment 11.

FIG. 10 shows a hydrogen spectrum of ionizable lipid 8-5-C8C10 prepared by Embodiment 12.

FIG. 11 shows a hydrogen spectrum of ionizable lipid 14-5-C8C10 prepared by Embodiment 13.

FIG. 12 shows a diagram of relative luciferase activity results of lipid nanoparticles of Embodiment 14 for cell transfection.

FIG. 13 shows a diagram of relative luciferase activity results of lipid nanoparticles having different neutral phospholipids of Embodiment 15 for cell transfection.

FIG. 14 shows a diagram of relative luciferase activity results of lipid nanoparticles having different component ratios of Embodiment 16 for cell transfection.

FIG. 15 shows a diagram of relative luciferase activity results of lipid nanoparticles having different nitrogen to phosphorus ratios of Embodiment 17 for cell transfection.

FIG. 16 shows a diagram of relative luciferase activity results of lipid nanoparticles transfected with different buffers of Embodiment 18 for cell transfection.

FIG. 17 shows a diagram of luciferase activity results of lipid nanoparticles of Embodiment 19 transfected into mice in vivo.

FIG. 18 shows an image of lipid nanoparticles of Embodiment 19 transfected into mice in vivo.

FIG. 19 shows a diagram of relative luciferase activity results of lipid nanoparticles of Embodiment 20 for cell transfection.

FIG. 20 shows a diagram of luciferase activity results of lipid nanoparticles of Embodiment 21 transfected into mice in vivo.

### DETAILED DESCRIPTION OF THE EMBODIMENTS

In order to further clarify the purpose, technical solution, and advantages of the present disclosure, the present disclosure will be further described in detail with reference to the accompanying drawings and embodiments. It should be understood that the specific embodiments described here are only used to explain the present disclosure and are not intended to limit the present disclosure.

The preparation method of the multi-tail type ionizable lipid of the present disclosure includes the following steps.

### (1) Synthesis of Hydrophobic Tail

The specific steps are as follows. 5 mmol of alkyl alcohol, 15 ml of N,N′-carbonyl diimidazole, 10 mmol of triethylamine (TEA), 20 ml of dichloromethane (DCM), and magnets are added in a 50 mL reaction tube in sequence. The reaction tube is placed in a heating tube at 40° C. and reacted for 24 hours until the reaction is complete. The reaction mixture is transferred into a separatory funnel, DCM (2×100 mL) and a saturated saline (2×100 mL) are added for extraction, and the extracted solution is washed with 1 M HCl (2×20 mL). An organic layer is collected and dried with anhydrous magnesium sulfate and filtered to obtain the product, which can proceed to the next reaction step without further purification.

5 mmol of the product obtained above, 10 mmol of amino alcohol, and 20 mL of DCM are added into a 50 mL reaction tube containing magnets. The reaction tube is placed in a heating jacket at 40° C. and reacted for 24 hours. When temperature of the reaction is cooled to room temperature, the reaction mixture is transferred into a separatory funnel, DCM (2×100 mL) and a saturated saline (2×100 mL) are added to perform extraction, and the extracted solution is washed with 1 M HCl (2×20 mL). An organic layer is collected and dried with anhydrous magnesium sulfate and filtered. A vacuum rotary evaporator is used for the products after filtering to remove the organic solvent. The product after removing the organic solvent is separated by a thin-layer chromatography column.

### (2) Synthesis of Linking Groups

5 mmol of the hydrophobic alkyl tail of the product synthesized above, 7.5 mmol of TEA, and 20 mL of DCM are added in a three necked flask containing magnets. The three necked flask is precooled in an ice bath for 30 minutes, and 6.25 mmol of acryloyl chloride (premixed in 10 mL of dichloromethane) is slowly added dropwise by utilizing a constant pressure funnel. After the acryloyl chloride is added dropwise, the ice bath is removed. The mixture is placed at room temperature and overnight reacted. Then the reacted mixture is diluted with DCM (30 mL) and washed with 1 M HCl (50 mL). An organic layer is dried by utilizing anhydrous magnesium sulfate and filtered to obtain a product, and the product is separated by a rapid chromatography column.

### (3) Reaction between Head Group and Tail Group

The synthesized alkyl tail with a chemical equivalent synthesized in above step (2) and 100 mg of amine are selected and sequentially added into a 3 mL reaction flask lined with tetrafluoroethylene. The mixture is heated at 90° C. for 48 hours and reacted to obtain a product. After the reaction is complete, the product can be directly subjected to cell transfection experiments or separated by a rapid chromatography column.

For the ionizable lipid library synthesized by the present disclosure, the reaction steps are simple and have mild conditions, and the ionizable lipid library can be prepared in large quantities within one week. This ionizable lipid can efficiently transfect mRNA and meet the delivery requirements of the new generation RNA vaccine. Transfection effect of the preferred ionizable lipid is comparable to, or even superior to, that of the several marketed lipid products.

### Embodiment 1

5 mmol of 3-nonanol, 15 mmol of N,N′-carbonyl diimidazole, 10 mmol of TEA, 20 mL of DCM, and magnets are added in a 50 mL reaction tube in sequence. The reaction tube is placed in a heating jacket at 40° C. and reacted for 24 hours, and the reaction progress is detected by utilizing Thin Layer Chromatography (TLC) until the reaction is complete. The reacted mixture is transferred into a separatory funnel, DCM (2×100 mL) and saturated saline (2×100 mL) are added for extraction, and the extracted solution is washed with IM HCl (2×20 mL). An organic layer is collected and dried by utilizing anhydrous magnesium sulfate and filtered, then a vacuum rotary evaporator is used to remove the organic solvent to obtain product A. The product A can proceed to the next step for reaction without further purification.

### Embodiment 2

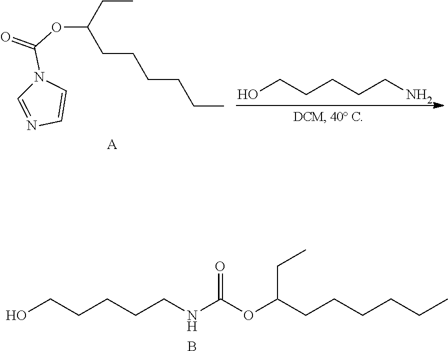

5 mmol of intermediate product A, 10 mmol of 5-amino-1-pentanol, and 20 mL of DCM are added sequentially into a 50 mL reaction tube containing magnets. The reaction tube is placed in a heating jacket at 40° C. and reacted for 24 hours. A TLC is used to detect the reaction progress until the reaction is complete. The reacted mixture is transferred into a separatory funnel, DCM (2×100 mL) and a saturated saline (2×100 mL) are added for extraction, and the extracted solution is washed with 1 M HCl (2×20 mL). An organic layer is collected and dried by utilizing anhydrous magnesium sulfate and filtered. Then a vacuum rotary evaporator is used to remove the organic solvent to obtain a product. The product is separated by utilizing a thin-layer chromatography column to obtain the target product B with a yield of 85%.

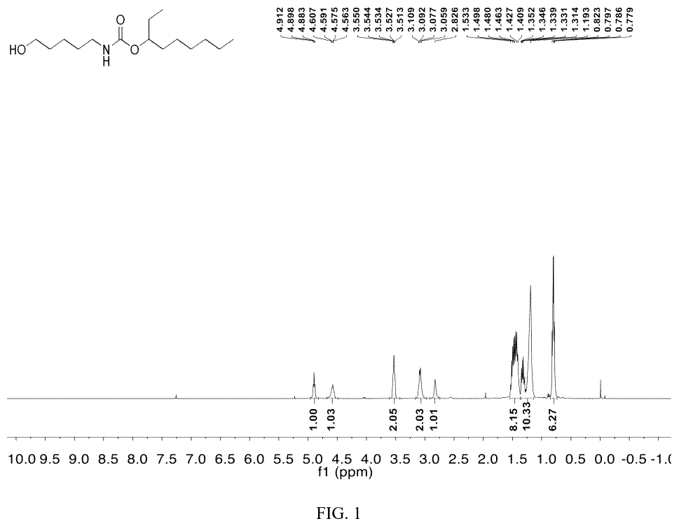

The hydrogen spectrum of the obtained product is shown in FIG. 1, and the hydrogen spectrum data is as follows:

¹H NMR (400 MHZ, CDCl₃): 4.90 (t, J=5.6 Hz, 1H), 4.61-4.56 (m, 1H), 3.55-3.51 (m, 2H), 3.11-3.06 (m, 2H), 2.83 (s, 1H), 1.53-1.41 (m, 8H), 1.35-1.19 (m, 10H), 0.82-0. 78 (m, 6H).

### Embodiment 3

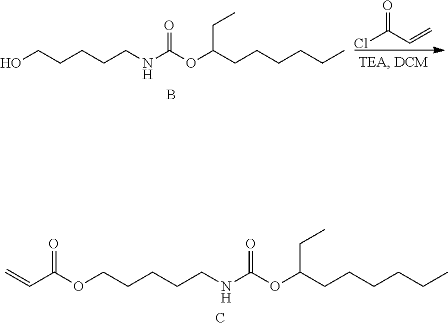

5 mmol of intermediate product B, 7.5 mmol of TEA, and 20 mL of DCM are added sequentially into a three necked flask containing magnets. The three necked flask is precooled in an ice bath for 30 minutes. 6.25 mmol of acryloyl chloride (premixed in 10 mL of DCM) is slowly added dropwise by utilizing a constant pressure funnel. After the acryloyl chloride is added dropwise, the ice bath is removed. The reacted solution is reacted at room temperature for 24 hours. A TLC is used to detect the reaction progress until the reaction is complete. The reacted solution is diluted by utilizing DCM (2×50 mL) and washed by utilizing 1 M HCl (2×20 mL). An organic layer is collected and dried by utilizing anhydrous magnesium sulfate and filtered. Then a vacuum rotary evaporator is used to remove the organic solvent to obtain a product. The product is separated by a thin-layer chromatography column to obtain the target product C with a yield of 90%.

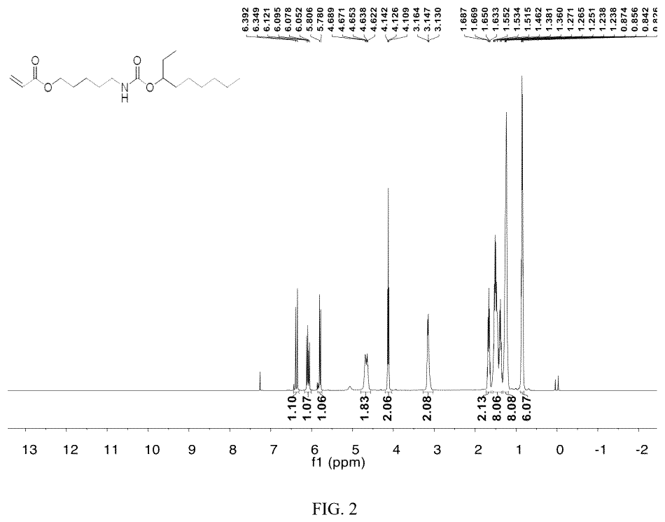

The hydrogen spectrum of the obtained product is shown in FIG. 2, and the hydrogen spectrum data is as follows:

¹H NMR (400 MHz, CDCl₃): 6.37 (d, J=17.2, 1H), 6.08 (q, J=6.8 Hz, 1H), 5.79 (d, J=10.4, 1H), 4.69-4.62 (m, 2H), 4.13 (t, J=6.4 Hz, 2H), 3.16-3.13 (m, 2H), 1.70-1.63 (m, 2H), 1.55-1.36 (m, 8H), 1.27-1.24 (m, 8H), 0.85 (dd, J=5.6 Hz, J=3.6 Hz, 6H).

### Embodiment 4

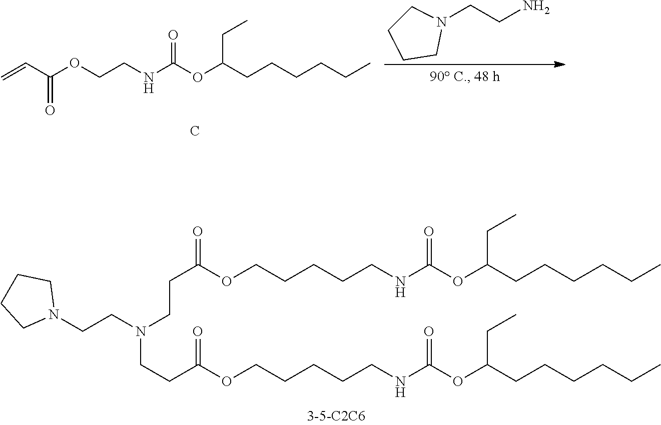

100 mg of 1-(2-aminoethyl) pyrrolidine and twice chemical equivalent of the intermediate product C are added into a 5 mL reaction flask containing magnets (with a tetrafluoroethylene liner on the cap of the reaction flask), and reacted at 90° C. for 48 hours. After the reaction is complete, the product is separated by a thin layer chromatography column to obtain ionizable lipid 3-5-C2C6.

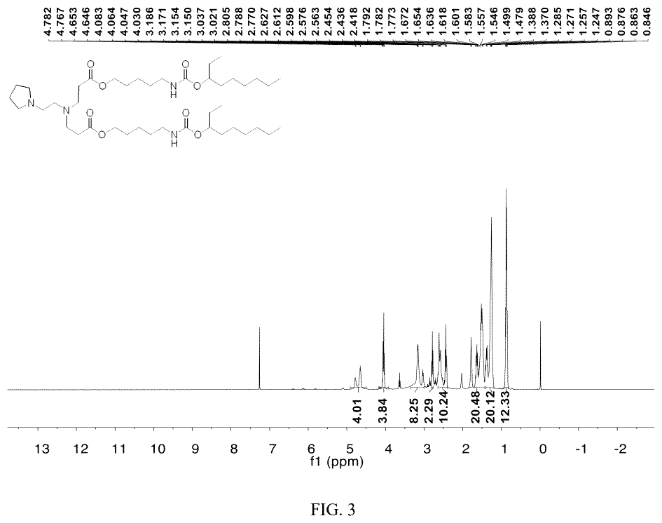

The hydrogen spectrum of the obtained product is shown in FIG. 3, and the hydrogen spectrum data is as follows:

¹H NMR (400 MHz, CDCl₃): 4.78-4.65 (m, 4H), 4.08-4.03 (m, 4H), 3.19-3.02 (m, 8H), 2.81-2.77 (m, 2H), 2.63-2.42 (m, 10H), 1.80-1.48 (m, 20H), 1.39-1.25 (m, 20H), 0.87 (dd, J=6.8 Hz, J=5.2 Hz, 12H).

### Embodiment 5

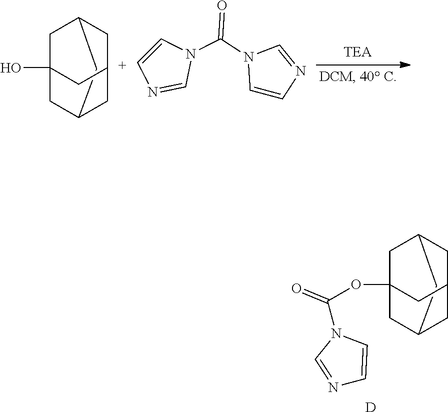

5 mmol of 1-adamantan, 15 mmol of N,N′-carbonyl diimidazole, 10 mmol of TEA, 20 mL of DCM, and magnets are added sequentially into a 50 mL reaction tube. The reaction tube is placed in a heating jacket at 40° C. and reacted for 24 hours. A TLC is used to detect the reaction progress until the reaction is complete. The reacted mixture is transferred into a separatory funnel, DCM (2×100 mL) and saturated saline (2×100 mL) are added for extraction, and the extracted solution is washed with 1 M HCl (2×20 mL). An organic layer is collected and dried by utilizing anhydrous magnesium sulfate and filtered. Then a vacuum rotary evaporator is used to remove the organic solvent. The product is separated by thin-layer chromatography column to obtain the target product D with a yield of 80%.

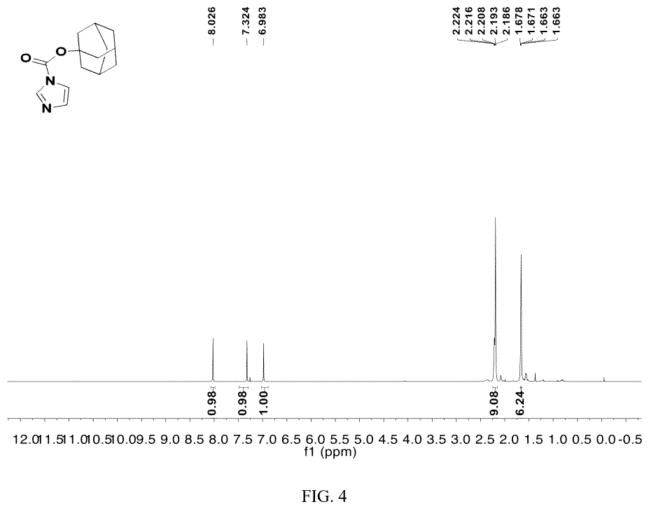

The hydrogen spectrum of the obtained product is shown in FIG. 4, and the hydrogen spectrum data is as follows:

¹H NMR (400 MHZ, CDCl₃): 8.03 (s, 1H), 7.32 (s, 1H), 6.98 (s, 1H), 2.22-2.19 (m, 9H), 1.68-1.66 (m, 6H).

### Embodiment 6

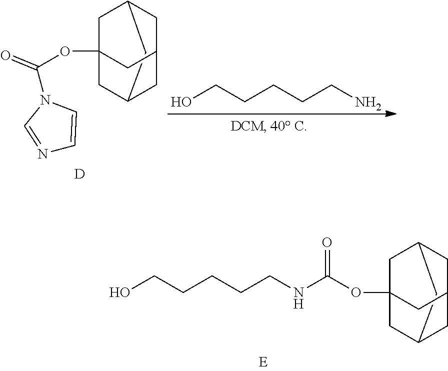

5 mmol of the intermediate product D, 10 mmol of 5-amino-1-pentanol, and 20 mL of DCM are added sequentially into a 50 mL reaction tube containing magnets. The reaction tube is placed in a heating jacket at 40° C. and reacted for 24 hours. A TLC is used to detect the reaction progress and until the reaction is complete. The reacted mixture is transferred into a separatory funnel, DCM (2×100 mL) and saturated saline (2×100 mL) are added for extraction, and the extracted solution is washed by utilizing 1 M HCl (2×20 mL). An organic layer is collected and dried by utilizing anhydrous magnesium sulfate and filtered. Then a vacuum rotary evaporator is used to remove the organic solvent. The product is separated by a thin-layer chromatography column to obtain the target product E with a yield of 76%.

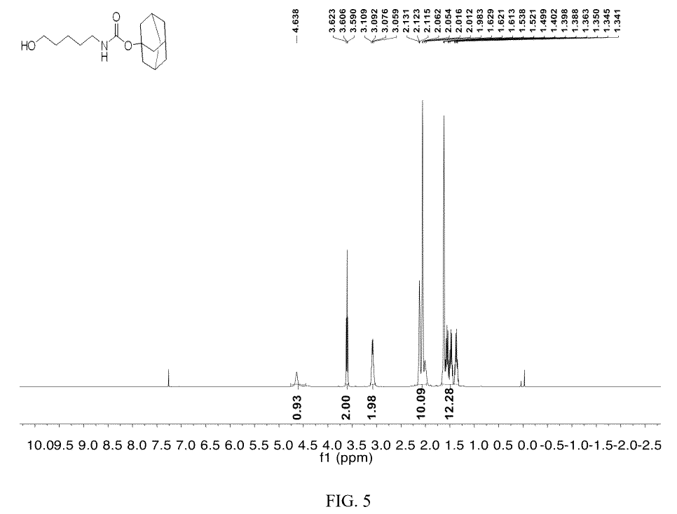

The hydrogen spectrum of the obtained product is shown in FIG. 5, and the hydrogen spectrum data is as follows:

1H NMR (400 MHZ, CDCl₃): 4.64 (s, 1H), 3.61 (t, J=6.8 Hz, 2H), 3.11-3.06 (m, 2H), 2.13-1.98 (m, 10H), 1.63-1.34 (m, 12H).

### Embodiment 7

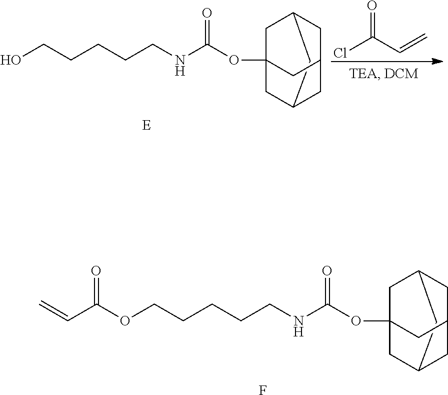

5 mmol of the intermediate product E, 7.5 mmol of TEA, and 20 mL of DCM are sequentially added into a three necked flask containing magnets. The three necked flask is precooled in an ice bath for 30 minutes, and 6.25 mmol of acryloyl chloride (premixed in 10 mL of DCM) is slowly added dropwise by utilizing a constant pressure funnel. After the acryloyl chloride is added dropwise, the ice bath is removed. The reacted solution is reacted at room temperature for 24 hours, and the reaction progress is detected by a TLC until the reaction is complete. DCM (2×50 mL) is used to diluted and the diluted solution is washed with 1 M HCl (2×20 mL). An organic layer is collected and dried by utilizing anhydrous magnesium sulfate and filtered. Then a vacuum rotary evaporator is used to remove the organic solvent. The product is separated by thin-layer chromatography column to obtain the target product F with a yield of 88%.

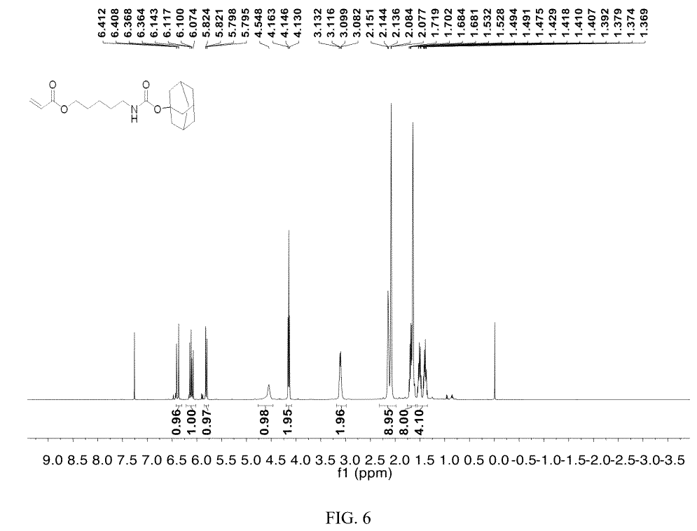

The hydrogen spectrum of the obtained product is shown in FIG. 6, and the hydrogen spectrum data is as follows:

¹H NMR (400 MHZ, CDCl₃): 6.39 (dd, J=16.0 Hz, J=1.6 Hz, 1H), 6.11 (q, J-6.8 Hz, 1H), 5.80 (dd, J=9.2 Hz, J=1.2 Hz, 1H), 4.55 (s, 1H), 4.15 (t, J-6.4 Hz, 2H), 3.13-3.08 (m, 2H), 2.15-2.08 (m, 9H), 1.72-1.68 (m, 8H), 1.53-1.37 (m, 4H).

### Embodiment 8

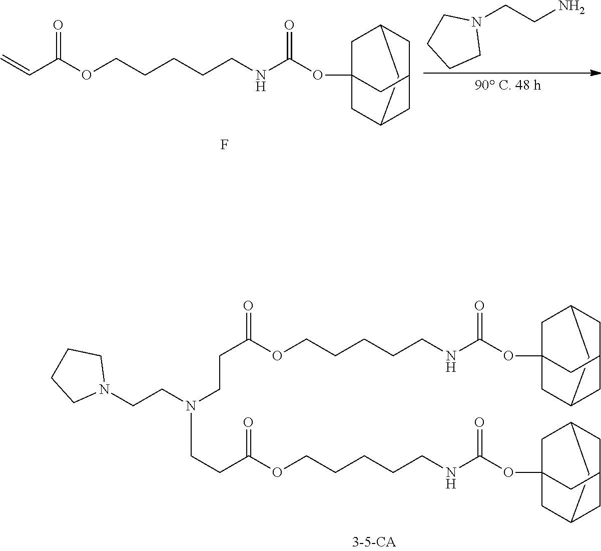

100 mg of 1-(2-aminoethyl) pyrrolidine and twice chemical equivalent of the intermediate product F are added into a 5 mL reaction flask containing magnets (with a tetrafluoroethylene liner on the cap of the flask). The reaction flask is placed at 90° C. and reacted for 48 hours. After the reaction is complete, the product is separated by a thin layer chromatography column to obtain ionizable lipid 3-5-CA.

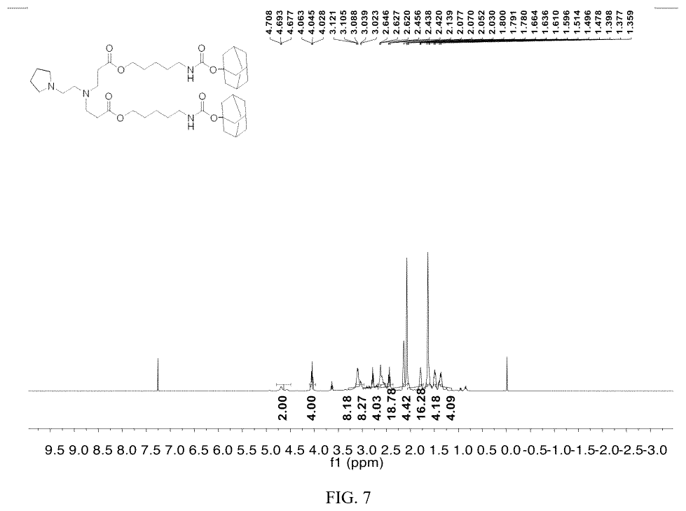

The hydrogen spectrum of the obtained product is shown in FIG. 7, and the hydrogen spectrum data is as follows:

¹H NMR (400 MHZ, CDCl₃): 4.71-4.68 (m, 2H), 4.05 (t, J=7.2 Hz, 4H), 3.12-3.02 (m, 8H), 2.65-2.62 (m, 8H), 2.46-2.42 (m, 4H), 2.14-2.03 (m, 18H), 1.80-1.78 (m, 4H), 1.66-1.60 (m, 16H), 1.51-1.48 (m, 4H), 1.40-1.36 (m, 4H).

### Embodiment 9

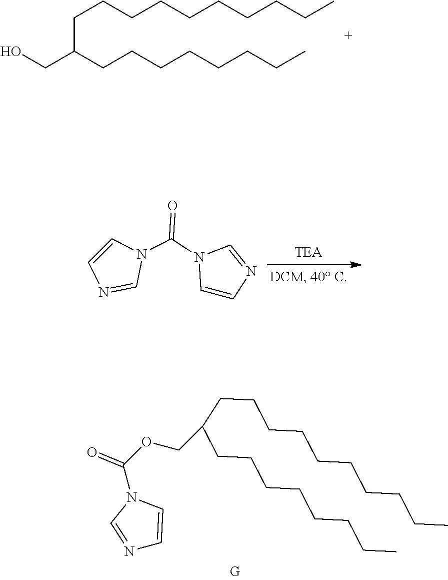 [SMILES: CCCCCCCCCCC(CO)CCCCCCCC ; O=C(n1ccnc1)n1ccnc1 ; CCCCCCCCCCC(CCCCCCCC)COC(=O)n1ccnc1]

5 mmol of 2-octyldodecanol, 15 mmol of N,N′-carbonyl diimidazole, 10 mmol of TEA, 20 mL of DCM, and magnets are added into a 50 mL reaction tube. The reaction tube is placed in a heating jacket at 40° C. and reacted for 24 hours. A thin-layer chromatography TLC is used to detect the reaction progress until the reaction is complete. The reacted mixture is transferred into a separatory funnel. DCM (2×100 mL) and saturated saline (2×100 mL) are added for extraction, and the extracted solution is washed by utilizing 1 M HCl (2×20 mL). An organic layer is collected and dried by utilizing anhydrous magnesium sulfate and filtered. Then a vacuum rotary evaporator is used to remove the organic solvent to obtain product G. The product G can proceed to the next step for reaction without further purification.

### Embodiment 10

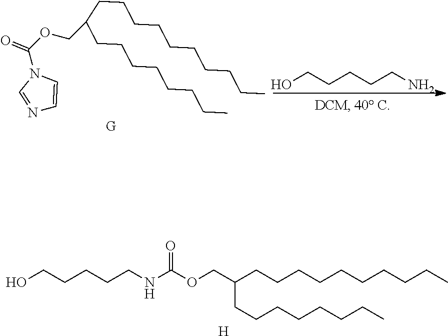

5 mmol of the intermediate product G, 10 mmol of 5-amino-1-pentanol, and 20 mL of DCM are added sequentially into a 50 mL reaction tube containing magnets. The reaction tube is placed in a heating jacket at 40° C. and reacted for 24 hours. A TLC is used to detect the reaction progress until the reaction is complete. The reacted mixture is transferred into a separatory funnel. DCM (2×100 mL) and saturated saline (2×100 mL) are added for extraction, and the extracted solution is washed by utilizing 1 M HCl (2×20 mL). An organic layer is collected and dried by utilizing anhydrous magnesium sulfate and filtered. Then a vacuum rotary evaporator is used to remove the organic solvent. The product is separated by thin-layer chromatography column to obtain the target product H with a yield of 76%.

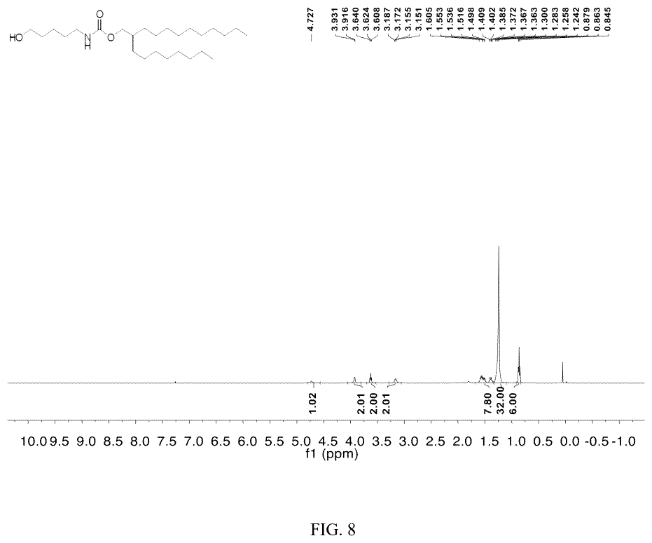

The hydrogen spectrum of the obtained product is shown in FIG. 8, and the hydrogen spectrum data is as follows:

¹H NMR (400 MHZ, CDCl₃): 4.73 (s, 1H), 3.93-3.92 (m, 2H), 3.62 (t, J=6.4 Hz, 2H), 3.19-3.15 (m, 2H), 1.61-1.36 (m, 8H), 1.30-1.24 (m, 32H), 0.88-0.85 (t, J=6.4 Hz, 6H).

### Embodiment 11

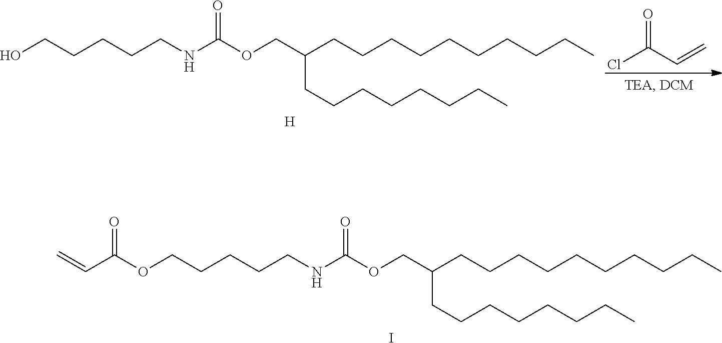

5 mmol of the intermediate product H, 7.5 mmol of TEA, and 20 mL of DCM are added sequentially into a three necked flask containing magnets. The three necked flask is precooled in an ice bath for 30 minutes. 6.25 mmol of acryloyl chloride (premixed in 10 mL of DCM) is slowly added dropwise by utilizing a constant pressure funnel. After the acryloyl chloride is added dropwise, the ice bath is removed. The reacted solution is placed at room temperature and reacted for 24 hours. The reaction progress is detected by utilizing a TLC until the reaction is complete. The reacted solution is diluted by utilizing DCM (2×50 mL) and the diluted solution is washed by utilizing 1 M HCl (2×20 mL). An organic layer is collected and dried by utilizing anhydrous magnesium sulfate and filtered. Then a vacuum rotary evaporator is used to remove the organic solvent. The product is separated by thin-layer chromatography column to obtain the target product I with a yield of 88%.

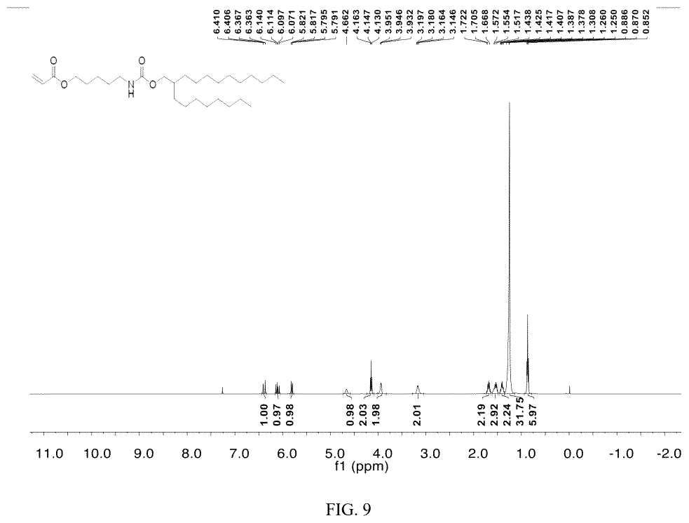

The hydrogen spectrum of the obtained product is shown in FIG. 9, and the hydrogen spectrum data is as follows:

¹H NMR (400 MHZ, CDCl₃): 6.38 (dd, J=15.6 Hz, J=1.6 Hz, 1H), 6.10 (q, J-6.8 Hz, 1H), 5.80 (dd, J=8.8 Hz, J=1.6 Hz, 1H), 4.66 (s, 1H), 4.15 (t, J=6.4 Hz, 2H), 3.95-3.93 (m, 2H), 3.20-3.15 (m, 2H), 1.72-1.67 (m, 2H), 1.57-1.52 (m, 2H), 1.44-1.38 (m, 2H), 1.31-1.25 (m, 32H), 0.87 (t, J-6.4 Hz, 6H).

### Embodiment 12

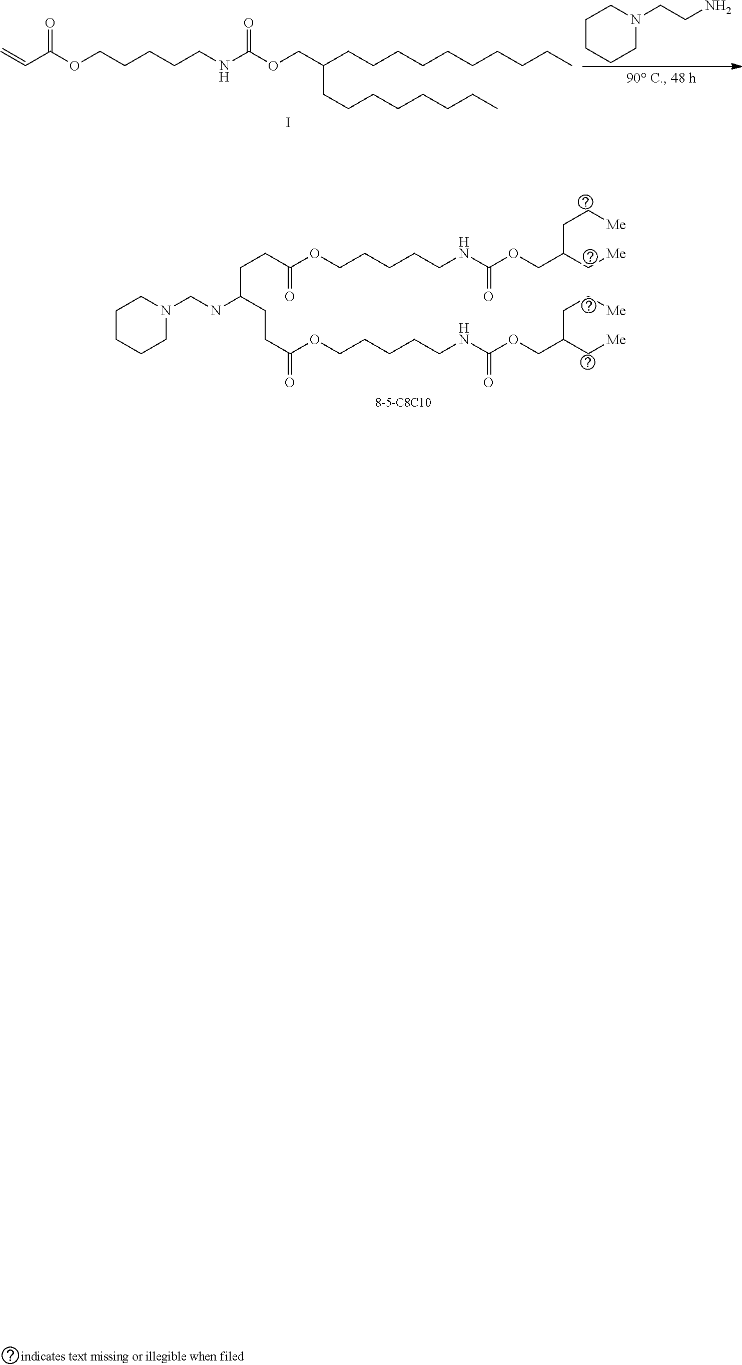

100 mg of 1-(2-aminoethyl) piperidine and twice chemical equivalent of the intermediate product I are added into a 5 mL reaction flask containing magnets (with a tetrafluoroethylene liner on the cap of the flask). The reaction flask is placed at 90° C. and reacted for 48 hours. After the reaction is complete, the product is separated by a thin layer chromatography column to obtain ionizable lipid 8-5-C8C10.

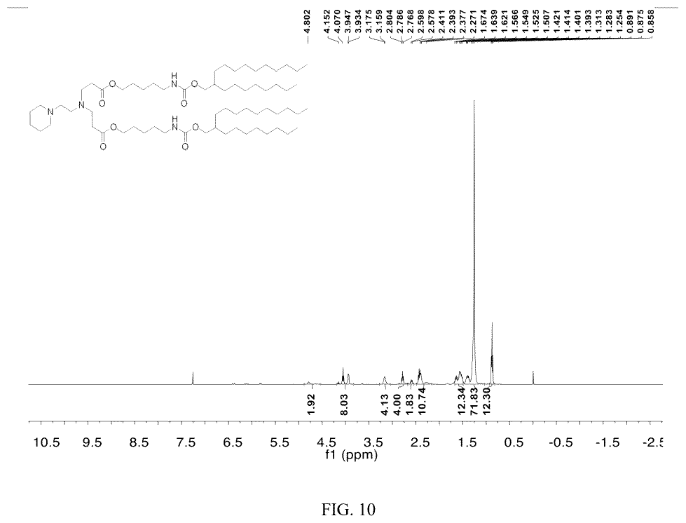

The hydrogen spectrum of the obtained product is shown in FIG. 10, and the hydrogen spectrum data is as follows:

¹H NMR (400 MHz, CDCl₃): 4.81 (s, 2H), 4.15-3.93 (m, 8H), 3.18-3.16 (m, 4H), 2.79 (t, J=7.2 Hz, 4H), 2.60-2.58 (m, 2H), 2.41-2.27 (m, 10H), 1.67-1.51 (m, 12H), 1.42-1.25 (m, 72H), 0.88 (t, J=6.4 Hz, 12H).

### Embodiment 13

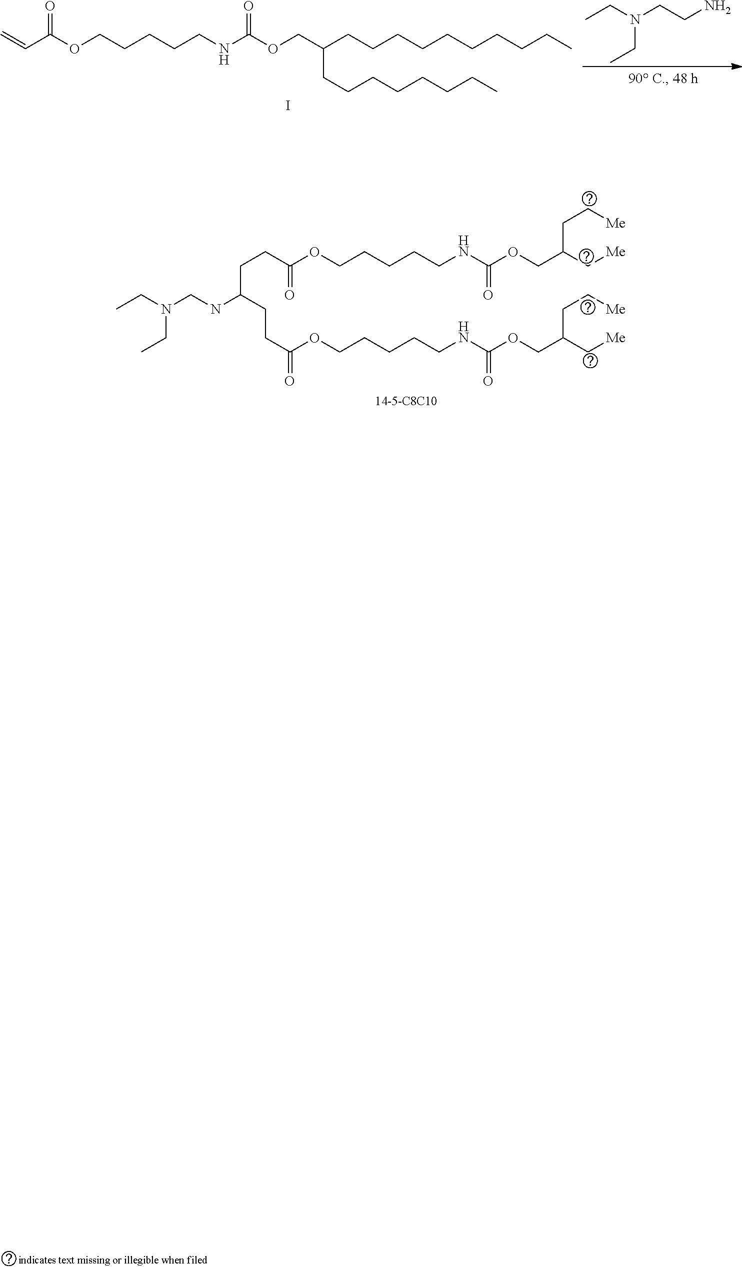

100 mg of N,N-diethylene diamine, twice chemical equivalent of the intermediate product I are added into a 5 mL reaction flask containing magnets (with a tetrafluoroethylene liner on the cap of the flask). The flask is placed at 90° C. and reacted for 48 hours. After the reaction is complete, the product is separated by a thin layer chromatography column to obtain ionizable lipid 14-5-C8C10.

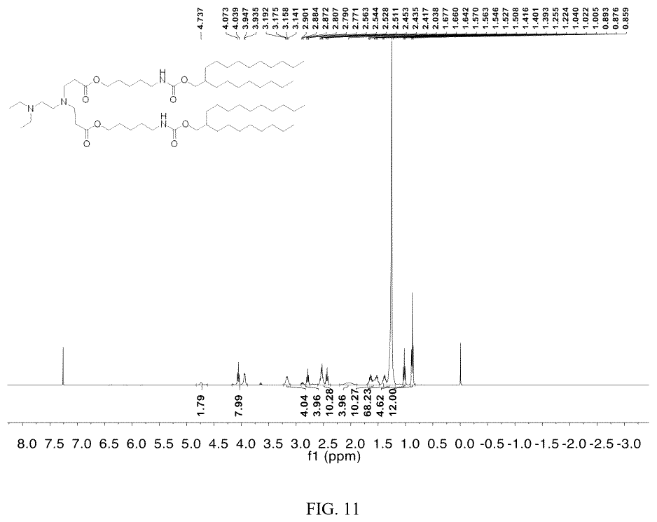

The hydrogen spectrum of the obtained product is shown in FIG. 11, and the hydrogen spectrum data is as follows:

¹H NMR (400 MHZ, CDCl₃): 4.74 (s, 2H), 4.07-3.94 (m, 8H), 3.20-3.14 (m, 4H), 2.91-2.78 (m, 4H), 2.56-2.42 (m, 10H), 2.04 (s, 4H), 1.68-1.51 (m, 10H), 1.42-1.22 (m, 68H), 1.02 (t, J=7.2 Hz, 4H), 0.88 (t, J=6.8 Hz, 12H).

The structure of the multi-tail type ionizable lipid synthesized by the present disclosure is as follows (for the synthesis methods of other multi-tail type ionizable lipids, please refer to Embodiments 1 to 13):

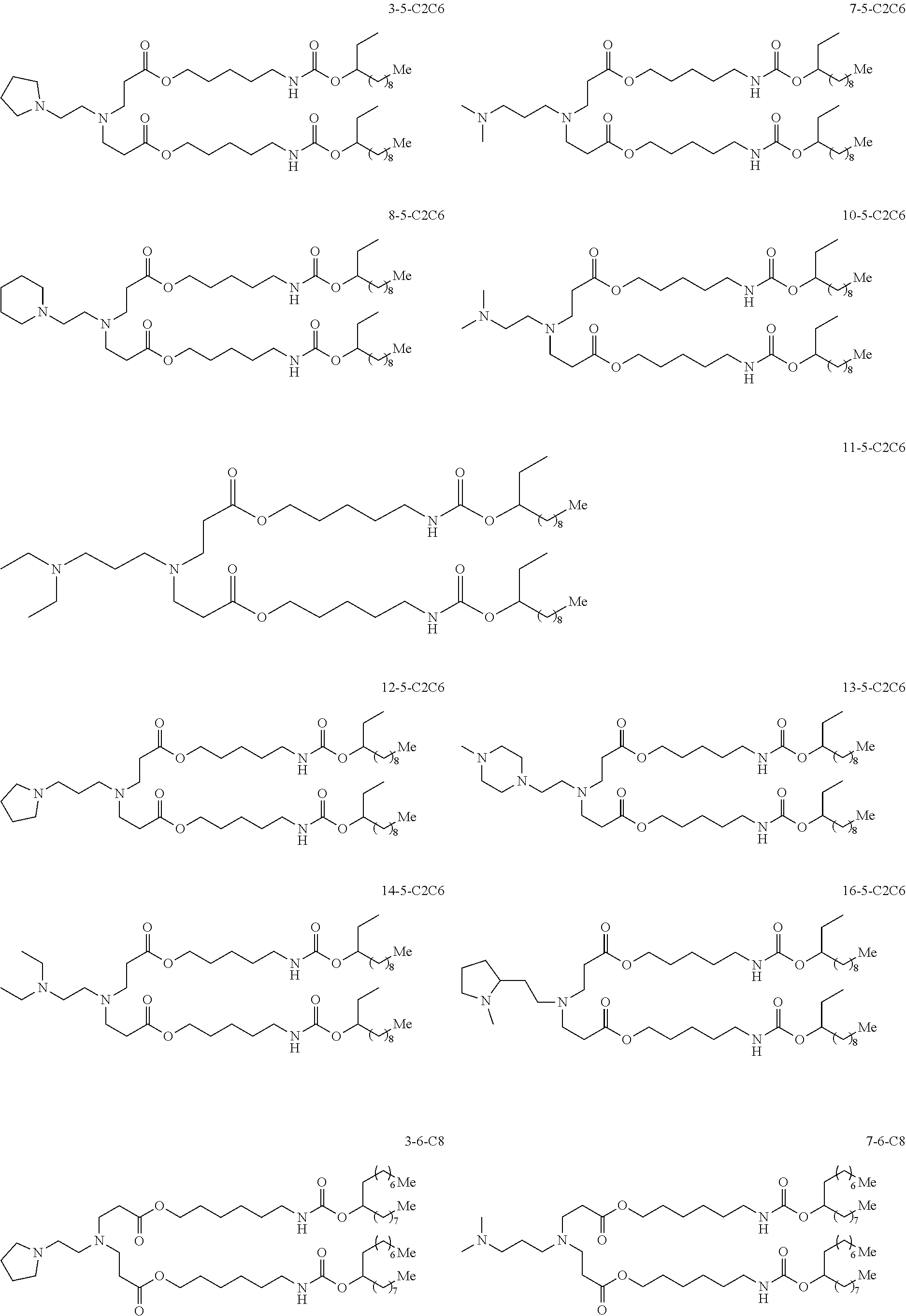

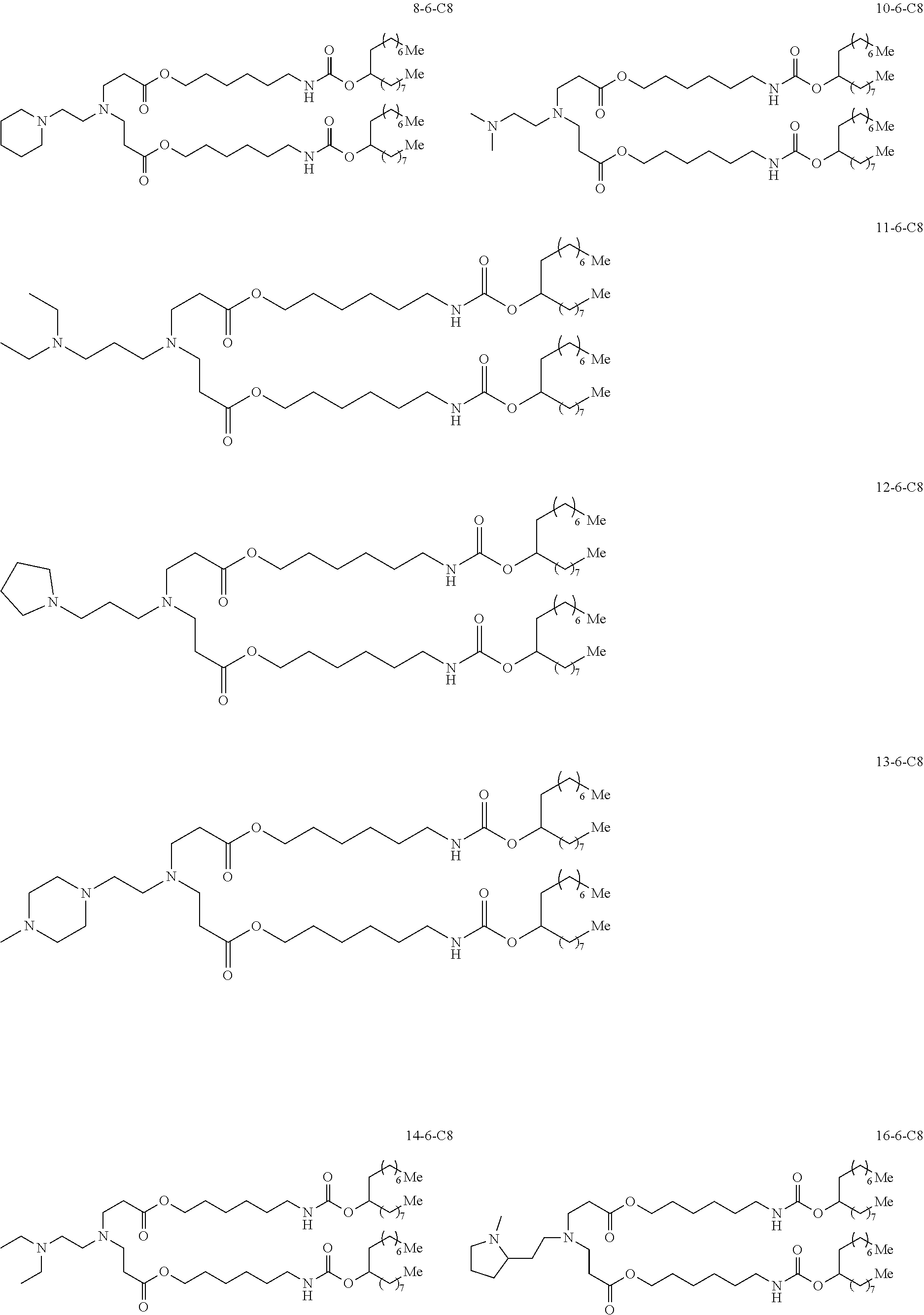

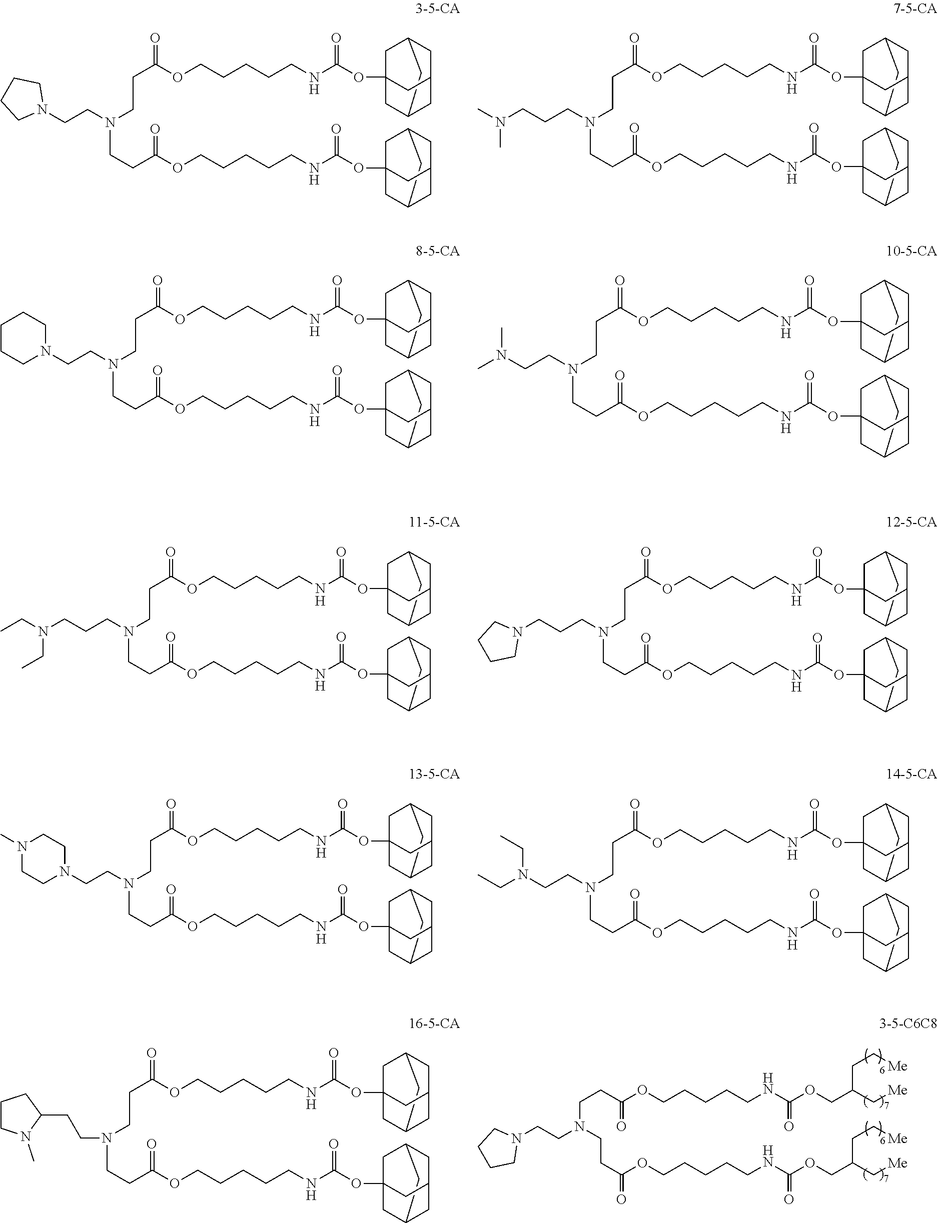

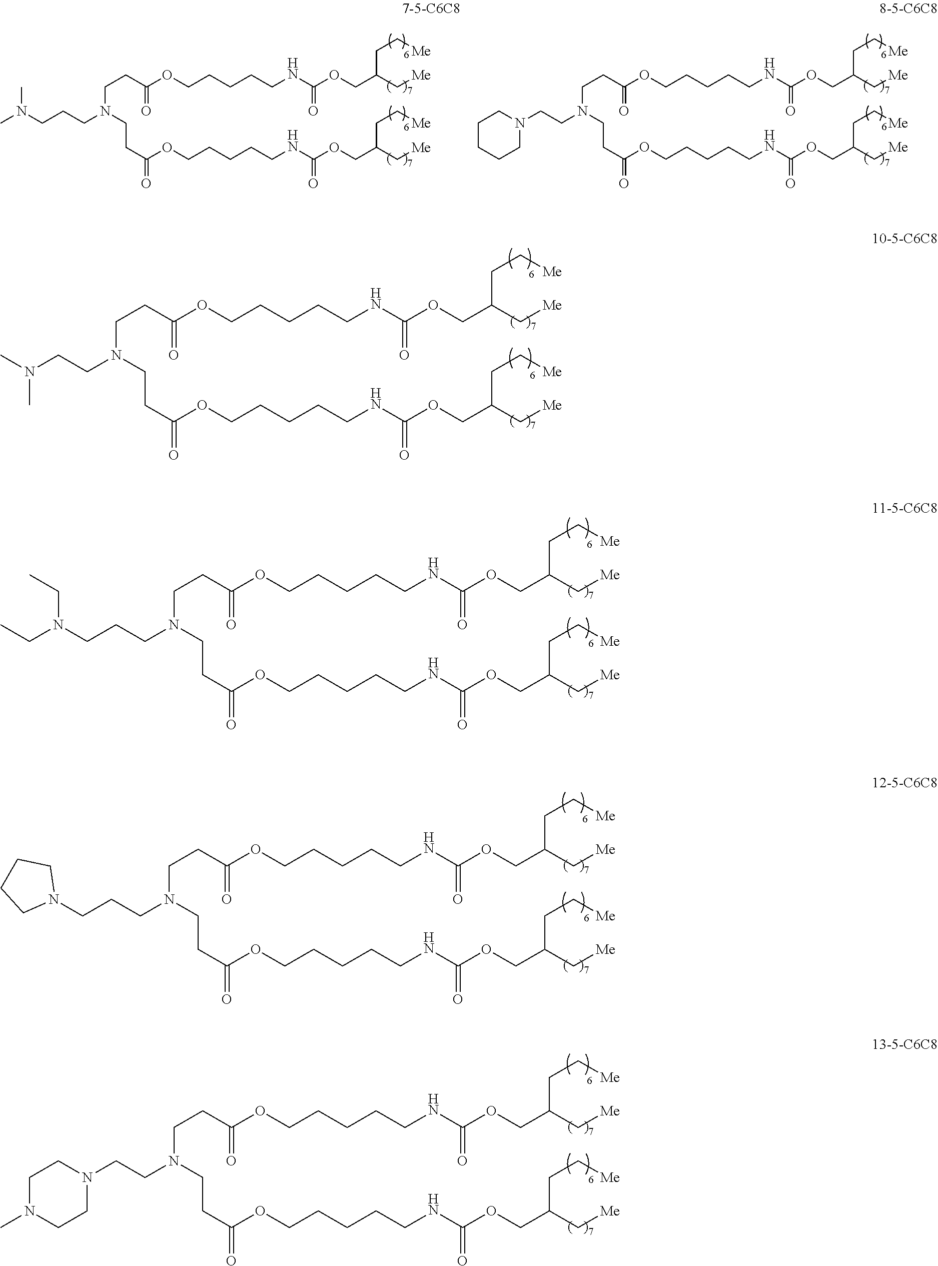

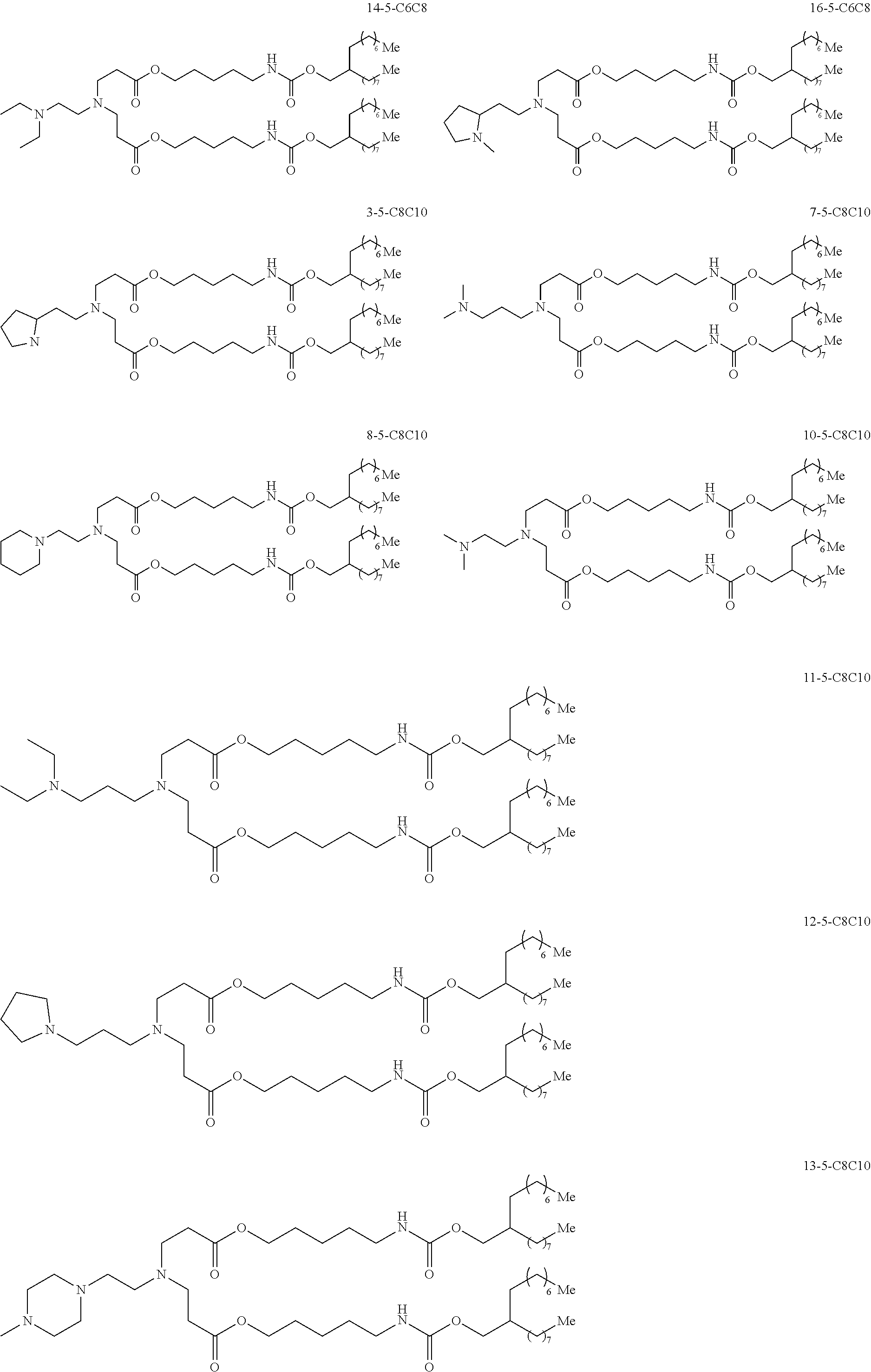

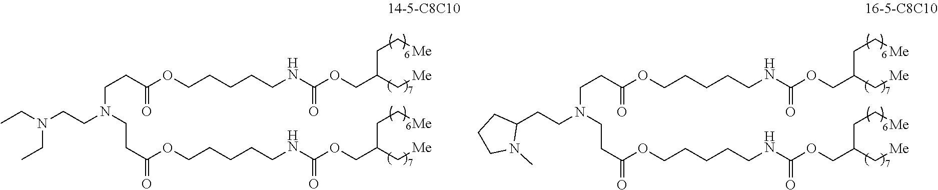

### Embodiment 14

Efficiency of LNP containing ionizable lipids in delivering self-diffusing RNA (repRNA-GFP-Luc) encoding green fluorescent protein (GFP) and firefly luciferase (Luc) is validated in 293T cell lines. Ionizable lipids such as 3-6-C8, 7-6-C8, 8-6-C8, 10-6-C8, 11-6-C8, 12-6-C8, 13-6-C8, 14-6-C8, 16-6-C8, 3-5-C2C6, 7-5-C2C6, 8-5-C2C6, 10-5-C2C6, 11-5-C2C6, 12-5-C2C6,13-5-C2C6, 14-5-C2C6, 16-5-C2C6, 3-5-CA, 7-5-CA, 8-5-CA, 10-5-CA, 11-5-CA, 12-5-CA, 13-5-CA, 14-5-CA, 16-5-CA, 3-5-C6C8, 7-5-C6C8, 8-5-C6C8, 10-5-C6C8, 11-5-C6C8, 12-5-C6C8,13-5-C6C8, 14-5-C6C8, 16-5-C6C8, 3-5-C8C10, 7-5-C8C10, 8-5-C8C10, 10-5-C8C10, 11-8C10, 12-5-C8C10, 13-5-C8C10, 14-5-C8C10, 16-5-C8C10 and commercial materials such as ALC-0315, SM-102 are used as delivery materials to express repRNA-GFP-Luc in cells, respectively.

### Specifical Steps:

### 1. Cell Culture

One day before the experiment, cultured 293T cell is seeded in a 96-well cell culture plate. A cell transfection experiment is started when a cell density of the 96-well cell culture plate growing to a ratio of around 70˜80%.

### 2. Preparation of Lipid Nanoparticles LNP-repRNA-GFP-Luc for Cell Transfection

Ionizable lipids such as 3-6-C8, 7-6-C8, 8-6-C8, 10-6-C8, 11-6-C8, 12-6-C8, 13-6-C8, 14-6-C8, 16-6-C8, 3-5-C2C6, 7-5-C2C6, 8-5-C2C6, 10-5-C2C6, 11-5-C2C6, 12-5-C2C6, 13-5-C2C6,14-5-C2C6, 16-5-C2C6, 3-5-CA, 7-5-CA, 8-5-CA, 10-5-CA, 11-5-CA, 12-5-CA, 13-5-CA, 14-5-CA, 16-5-CA, 3-5-C6C8, 7-5-C6C8, 8-5-C6C8, 10-5-C6C8, 11-5-C6C8, 12-5-C6C8, 13-5-C6C8, 14-5-C6C8, 16-5-C6C8, 3-5-C8C10, 7-5-C8C10, 8-5-C8C10, 10-5-C8C10, 11-8C10, 12-5-C8C10, 13-5-C8C10, 14-5-C8C10, and 16-5-C8C10 with a concentration of 10 mg/mL, Di stearoyl phosphatidylcholine (DSPC) with a concentration of 3 mg/mL, Cholesterol (Cholesterol) with a concentration of 6 mg/mL, and DSPE-PEG with a concentration of 1 mg/mL are dissolved in anhydrous ethanol in sequence, and mixed evenly in a molar ratio of ionizable lipids: Cholesterol: DSPC: DSPE-PEG=40:48:10:2. At the same time, an appropriate amount of repRNA-GFP-Luc is absorbed and dissolved in sodium acetate buffer (a volume of sodium acetate buffer is twice total volume of the lipid mixture, and pH=5.2˜5.3). Then the mRNA buffer and the lipid mixture solution are quickly mixed and incubated at room temperature for 15 minutes, and assembled to be a stable LNP (a LNP with 150 ng repRNA-GFP-Luc transfected per well). The LNP is diluted by utilizing twice volume of sterile PBS. The diluted LNP is added into a 96-well cell culture plate for transfection. The nitrogen phosphorus ratio between ionizable lipid and mRNA is 24:1, which is the molar ratio between protonated amino groups and phosphate groups on mRNA (the same below).

Positive control group: commercial lipids ALC-0315 and SM-102 are used and assembled to be the LNP according to the disclosed preparation method. The specific operation is as follows. ALC-0315 or SM-102 with a concentration of 5 mg/mL, DSPC with a concentration of 1.5 mg/mL, Cholesterol with a concentration of 3 mg/mL, and ALC-0159 or DMG-PEG2000 with a concentration of 1 mg/mL are dissolved in anhydrous ethanol in sequence, and mixed evenly in a molar ratio of ALC-0315: Cholesterol: DSPC: ALC-0159=46.3:42.7:9.4:1.6 or SM-102: Cholesterol: DSPC: DMG-PEG2000-50:38.5:10:1.5. At the same time, an appropriate amount of repRNA-GFP-Luc is adsorbed and dissolved in sodium citrate buffer (the volume of sodium citrate buffer is three times total volume of the lipid mixture, and pH=4.0). Then the mRNA buffer and the lipid mixture solution are mixed rapidly and incubated at room temperature for 15 minutes to assemble to be a stable LNP (a LNP with 150 ng repRNA-GFP-Luc transfected per well). The LNP is diluted by utilizing twice volume of sterile PBS and added to a 96-well cell culture plate for transfection. The nitrogen to phosphorus ratio of ALC-0315 or SM-102 to mRNA is 6:1.

Negative control group: the 293T cells are normally cultured, and added with unencapsulated repRNA-GFP-Luc.

### 3. Analysis of Cell Transfection Efficiency

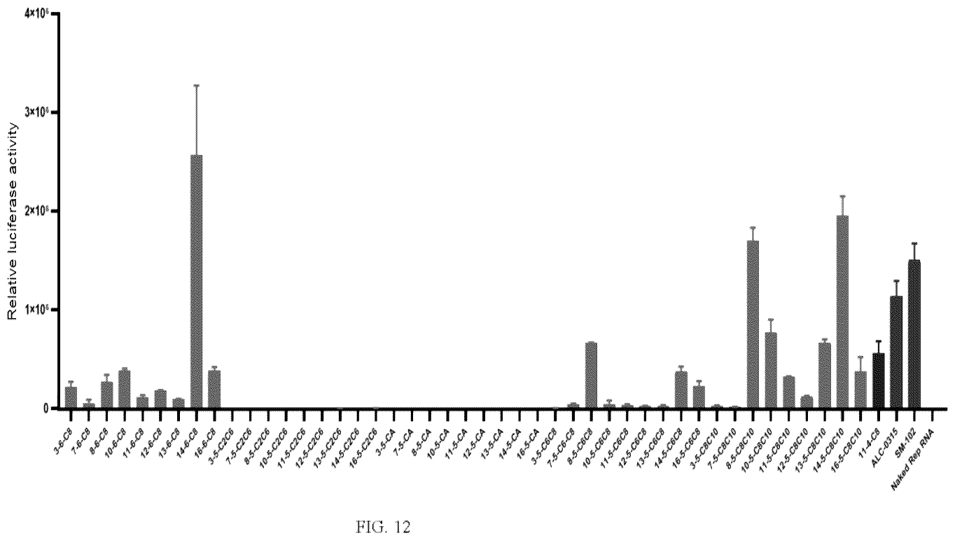

After 36 hours of cell transfection, the expression of green fluorescent protein is detected by utilizing a fluorescence microscope. The culture medium of the 96-well culture plate is completely sucked out. Cell lysis solution is added into the 96-well culture plate, and the cells are lysed on ice for 30 minutes. After centrifugation, the supernatant is extracted and transferred to a white 96-well detection plate. A firefly luciferase substrate is added on the white 96-well detection plate and an enzyme-linked immunosorbent assay (ELISA) reader is used to detect the firefly luciferase content (chemiluminescence). Relative luciferase activity results are shown in FIG. 12. The results show that the ionizable lipid synthesized by the present disclosure can greatly enhance the transfection efficiency of self-amplifying RNA. When the tail of the ionizable lipid is 6-C8, 5-C6C8, and 5-C8C10, the RNA expression efficiency is highest. When the tail is 5-C2C6 and 5-CA, the RNA expression efficiency is weaker. The transfection efficiency of the lipids represented by 14-6-C8, 8-5-C8C10, and 14-5-C8C10 is superior to commercial lipids ALC-0315 and SM-102, with an efficiency increase of about 2˜3 times, which verifies the rationality and efficiency of the overall chemical structure of the ionizable lipid designed by the present disclosure.

### Embodiment 15

Efficiency of LNP containing ionizable lipids in delivering self-diffusing RNA (repRNA-GFP-Luc) encoding green fluorescent protein and firefly luciferase is verified in 293T cell lines. Auxiliary lipids of LNP are optimized by utilizing ionizable lipids 14-6-C8, 8-5-C8C10, 14-5-C8C10.

### Specifical Steps:

1. Referring to Embodiment 14, the difference is that in Embodiment 15, the ionizable lipids used in the experimental group are: 14-6-C8, 8-5-C8C10, 14-5-C8C10. Ionizable lipids with a concentration of 10 mg/mL, dioleoyl phosphatidylethanolamine (DOPE), DSPC or dioleoyl phosphatidylcholine (DOPC) with a concentration of 3 mg/mL, Cholesterol with a concentration of 6 mg/mL, and DSPE-PEG with a concentration of 1 mg/mL are dissolved in anhydrous ethanol in sequence. The usage ratio of ionizable lipids 14-6-C8, 8-5-C8C10, or 14-5-C8C10: Cholesterol: DOPE, DSPC or DOPC: DSPE-PEG=40:48:10:2.
2. Analysis of Cell Transfection Efficiency

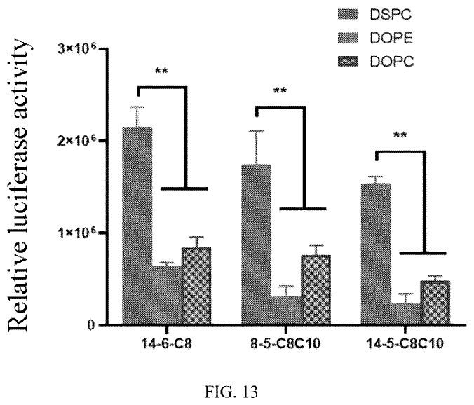

After transfection for 36 hours, the culture medium of the 96-well cell culture plate is completely sucked out, and a cell lysis solution is added on the 96-well cell culture plate and the cells are lysed on ice for 30 minutes. After centrifugation, the supernatant is extracted and transferred to a white 96-well detection plate. Firefly luciferase substrate is added on the white 96-well detection plate, and the firefly luciferase content (chemiluminescence) is detected by utilizing an enzyme-linked immunosorbent assay (ELISA) reader. The relative luciferase activity results are shown in FIG. 13. The results show that the chemical structure of auxiliary lipids greatly affects RNA delivery efficiency. When the auxiliary lipid is DSPC, the delivery efficiency of the three ionizable lipids is significantly better than DOPE or DOPC, so DSPC is the preferred auxiliary lipid.

### Embodiment 16

Efficiency of LNP containing ionizable lipids in delivering repRNA-GFP-Luc is verified in 293T cell lines. The proportion of each component in LNP is optimized by utilizing ionizable lipids 14-6-C8, 8-5-C8C10, 14-5-C8C10.

### Specifical Steps:

1. Referring to Embodiment 14, the difference is that in Embodiment 16, the ionizable lipids used in the experimental group are: 14-6-C8, 8-5-C8C10, 14-5-C8C10. Ionizable lipids with a concentration of 10 mg/mL, DSPC with a concentration of 3 mg/mL, Cholesterol with a concentration of 6 mg/mL, and DSPE-PEG with a concentration of 1 mg/mL are dissolved in anhydrous ethanol in sequence. They are mixed by utilizing five different molar ratios. Ratio A is that ionizable lipid: Cholesterol: DSPC: DSPE-PEG=40:48:10:2; Ratio B is that ionizable lipid: Cholesterol: DSPC: DSPE-PEG=30:28.5:10:0.75; Ratio C is that ionizable lipid: Cholesterol: DSPC: DSPE-PEG=50:38.5:10:1.5; Ratio D is that ionizable lipid: Cholesterol: DSPC: DSPE-PEG=35:46:16:2.5; and Ratio E is that ionizable lipid: Cholesterol: DSPC: DSPE-PEG=46.3:42.7:9.4:1.6
2. Analysis of Cell Transfection Efficiency

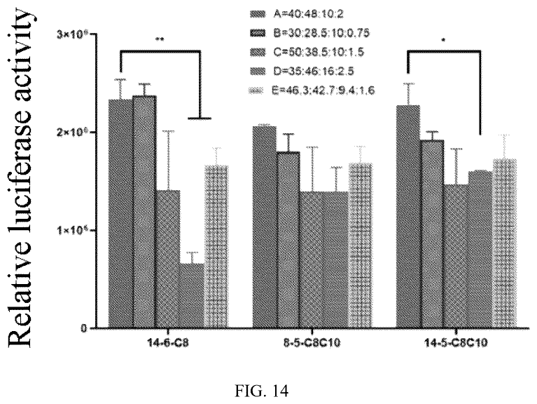

After transfection for 36 hours, the culture medium of the 96-well cell culture plate is completely sucked out. Cell lysis solution is added on the 96-well cell culture plate, and the cells are lysed on ice for 30 minutes. After centrifugation, the supernatant is extracted and transferred to a white 96-well detection plate. Firefly luciferase substrate is added on the white 96-well detection plate, and the firefly luciferase content (chemiluminescence) is detected by utilizing an enzyme-linked immunosorbent assay (ELISA) reader. The relative luciferase activity results are shown in FIG. 14. The results show that the molar ratio among components of LNP also affects RNA delivery efficiency to some extent. The optimal ratio is Ratio A: ionizable lipid: Cholesterol: DSPC: DSPE-PEG=40:48:10:2.

### Embodiment 17

Efficiency of LNP containing ionizable lipids in delivering repRNA-GFP-Luc is verified in 293T cell lines. A nitrogen phosphorus ratio of LNP is optimized by utilizing ionizable lipids 14-6-C8, 8-5-C8C10, 14-5-C8C10.

### Specifical Steps:

1. Referring to Embodiment 14, the difference is that in Embodiment 17, the ionizable lipids used in the experimental group are: 14-6-C8, 8-5-C8C10, 14-5-C8C10. Ionizable lipids with a concentration of 10 mg/mL, DSPC with a concentration of 3 mg/mL, Cholesterol with a concentration of 6 mg/mL, and DSPE-PEG with a concentration of 1 mg/mL are dissolved in anhydrous ethanol. The usage ratio is ionizable lipids 14-6-C8, 8-5-C8C10 or 14-5-C8C10: Cholesterol: DSPC: DSPE-PEG=40:48:10:2. The nitrogen phosphorus ratio of LNP is 12:1, 18:1, 24:1, and 32:1, respectively.
2. Analysis of Cell Transfection Efficiency

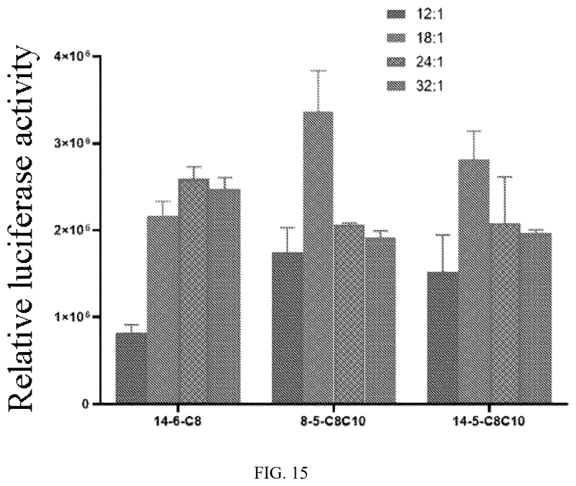

After transfection for 36 hours, the culture medium of the 96-well cell culture plate is completely sucked out. Cell lysis solution is added into the 96-well cell culture plate, and the cells are lysed on ice for 30 minutes. After centrifugation, the supernatant is extracted and transferred to a white 96-well detection plate. Firefly luciferase substrate is added on the white 96-well detection plate and the firefly luciferase content (chemiluminescence) is detected by utilizing an enzyme-linked immunosorbent assay (ELISA) reader. The relative luciferase activity results are shown in FIG. 15. The results show that the transfection efficiency of repRNA-GFP-Luc is optimal when the nitrogen to phosphorus ratio is 18:1.

### Embodiment 18

Efficiency of LNP containing ionizable lipids in delivering repRNA-GFP-Luc is verified in 293T cell lines. The buffer formulation of LNP is optimized by utilizing ionizable lipids 14-6-C8, 8-5-C8C10, 14-5-C8C10.

### Specifical Steps:

1. Referring to Embodiment 14, the difference is that in Embodiment 18, the ionizable lipids used in the experimental group are: 14-6-C8, 8-5-C8C10, 14-5-C8C10. Ionizable lipids with a concentration of 10 mg/mL, DSPC with a concentration of 3 mg/mL, Cholesterol with a concentration of 6 mg/mL, and DSPE-PEG with a concentration of 1 mg/mL are dissolved in anhydrous ethanol. The usage ratio is 14-6-C8, 8-5-C8C10 or 14-5-C8C10: Cholesterol: DSPC: DSPE-PEG=40:48:10:2. The solution of premixed RNA is a sodium acetate buffer or a sodium citrate buffer, and the nitrogen phosphorus ratio for preparing LNP is 18:1.
2. Analysis of Cell Transfection Efficiency

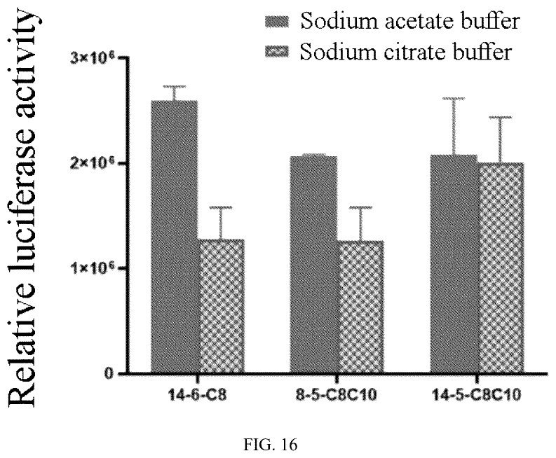

After transfection for 36 hours, the culture medium of the 96-well cell culture plate is completely sucked out. Cell lysis solution is added on the 96-well cell culture plate, and the cells are lysed on ice for 30 minutes. After centrifugation, the supernatant is extracted and transferred to a white 96-well detection plate. Firefly luciferase substrate is added on the white 96-well detection plate, and the firefly luciferase content (chemiluminescence) is detected by utilizing an enzyme-linked immunosorbent assay (ELISA) reader. The relative luciferase activity results are shown in FIG. 16. The results show that the RNA delivery efficiency is better when the buffer used for preparing nanoparticles is sodium acetate buffer than sodium citrate buffer. Therefore, the sodium acetate buffer is preferred.

### Embodiment 19

LNP containing ionizable lipids 14-6-C8, 8-5-C8C10, 10-5-C8C10, 14-5-C8C10, and commercial lipids SM-102 and ALC-0315 are used to deliver self-amplifying RNA (repRNA-Luc) encoding firefly luciferase in Balb/c mice. The expression of the gene luciferase is detected and reported by utilizing an in vivo imaging system (IVIS) on days 2, 5, 7, 10, 12, and 15 after intramuscular injection.
1. For specific steps, refer to Embodiment 14. The difference is that in Embodiment 19, the ionizable lipids used in the experimental group are: 14-6-C8, 8-5-C8C10, 10-5-C8C10, 14-5-C8C10. Ionizable lipids with a concentration of 10 mg/mL, DSPC with a concentration of 6 mg/mL, Cholesterol with a concentration of 12 mg/mL, and DSPE-PEG with a concentration of 5 mg/mL are dissolved in anhydrous ethanol. An appropriate amount of repRNA-Luc is adsorbed and dissolved in a sodium acetate buffer (volume of the sodium acetate buffer is twice total volume of the lipid mixture, and pH=5.3). Buffer containing repRNA-Luc is adsorbed and added into the lipid mixture ethanol solution, and quickly mixed evenly to assemble to be the LNP. The mixed solution is incubated at room temperature for 15 minutes, and dialyzed in PBS by utilizing a dialysis bag (MWCO=14000 MW) for 1 hour, and then an intramuscular injection (LNP containing 1.5 ug repRNA Luc is injected per injection) is performed. The usage ratio is ionizable lipids 14-6-C8, 8-5-C8C10, 10-5-C8C10, or 14-5-C8C10: Cholesterol: DSPC: DSPE-PEG=40:48:10:2. The solution of premixed RNA is a sodium acetate buffer, and the nitrogen phosphorus ratio for preparing LNP is 18:1.

Positive control group: commercial lipids ALC-0315 and SM-102 are used to assemble the corresponding positive control LNP according to the disclosed preparation method. The specific operation is as follows. ALC-0315 with a concentration of 5 mg/mL, DSPC with a concentration of 1.5 mg/mL, Cholesterol with a concentration of 3 mg/mL, and ALC-0159 or DMG-PEG2000 with a concentration of 1 mg/mL are dissolved in anhydrous ethanol. They are mixed uniformly according to a molar ratio of ALC-0315: Cholesterol: DSPC: ALC-0159=46.3:42.7:9.4:1.6, or a molar ratio of SM-102: Cholesterol: DSPC: DMG-PEG2000=50:38.5:10:1.5. At the same time, an appropriate amount of repRNA-Luc is adsorbed and dissolved in a sodium citrate buffer (volume of the sodium citrate buffer is three times total volume of the lipid mixture, and pH=4.0). Then the buffer containing mRNA and the lipid mixture solution are mixed rapidly, and incubated at room temperature for 15 minutes to assemble to be the LNP. The LNP is dialyzed in PBS for 1 hour by utilizing a dialysis bag (MWCO=14000 MW) and used for intramuscular injection (LNP containing 1.5 ug repRNA-Luc is injected per injection). The nitrogen to phosphorus ratio of ALC-0315 or SM-102 to mRNA is 6:1.
2. Analysis of In Vivo Imaging Results

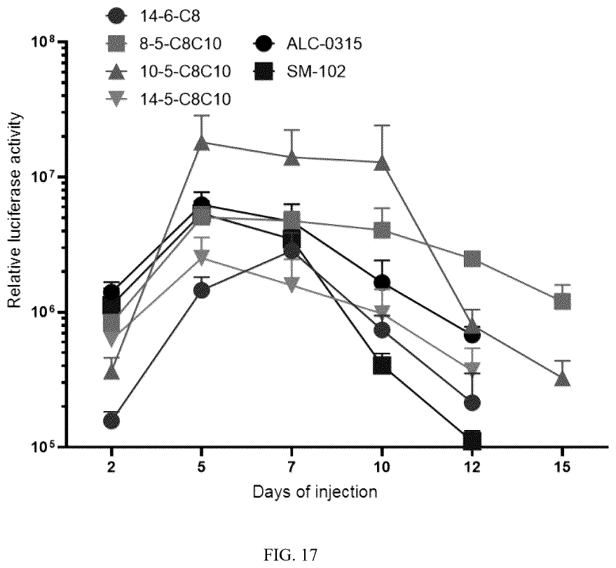

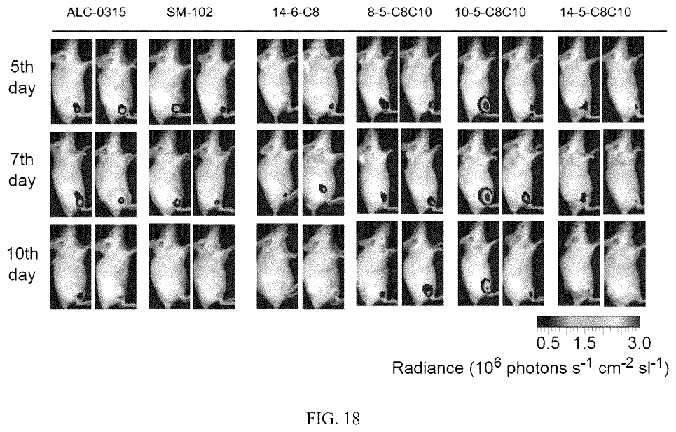

The IVIS results (FIGS. 17 to 18) show that from the second day of intramuscular injection, the expression intensity of the ionizable lipid 10-5-C8C10 of the present disclosure is better than that of the commercial lipids SM-102 and ALC-0315, and the decrease in expression level of 8-5-C8C10 in vivo is slower than that of commercial lipids.

According to literature reports, the expression value of self-amplifying RNA reaches its peak 7-10 days after injection, while ordinary mRNA reaches its peak 48 hours after injection, indicating that the self-amplifying RNA delivered by the ionizable lipid of the present disclosure has a longer expression time and a higher expression level, which can bring more efficient and lasting immune effects in mRNA vaccine applications.

### Embodiment 20

Efficiency of LNP containing ionizable lipids in delivering circular RNA (circRNA-Luc) encoding firefly luciferase is verified in 293T cell lines. Ionizable lipids 3-6-C8, 7-6-C8, 8-6-C8, 10-6-C8, 11-6-C8, 12-6-C8, 13-6-C8, 14-6-C8, 16-6-C8, 3-5-C2C6, 7-5-C2C6, 8-5-C2C6, 10-5-C2C6, 11-5-C2C6, 12-5-C2C6, 13-5-C2C6, 14-5-C2C6, 16-5-C2C6, 3-5-CA, 7-5-CA, 8-5-CA, 10-5-CA, 11-5-CA, 12-5-CA, 13-5-CA, 14-5-CA, 16-5-CA, 3-5-C6C8, 7-5-C6C8, 8-5-C6C8, 10-5-C6C8, 11-5-C6C8, 12-5-C6C8, 13-5-C6C8, 14-5-C6C8, 16-5-C6C8, 3-5-C8C10, 7-5-C8C10, 8-5-C8C10, 10-5-C8C10, 11-8C10, 12-5-C8C10, 13-5-C8C10, 14-5-C8C10, 16-5-C8C10 and commercial lipids ALC-0315 and SM-102 are used as delivery materials to express circRNA-Luc in cells.

### Specifical Steps:

1. Referring to Embodiment 14, the difference is that in Embodiment 20, repRNA-GFP-Luc is replaced with circRNA-Luc.
2. Analysis of Cell Transfection Efficiency

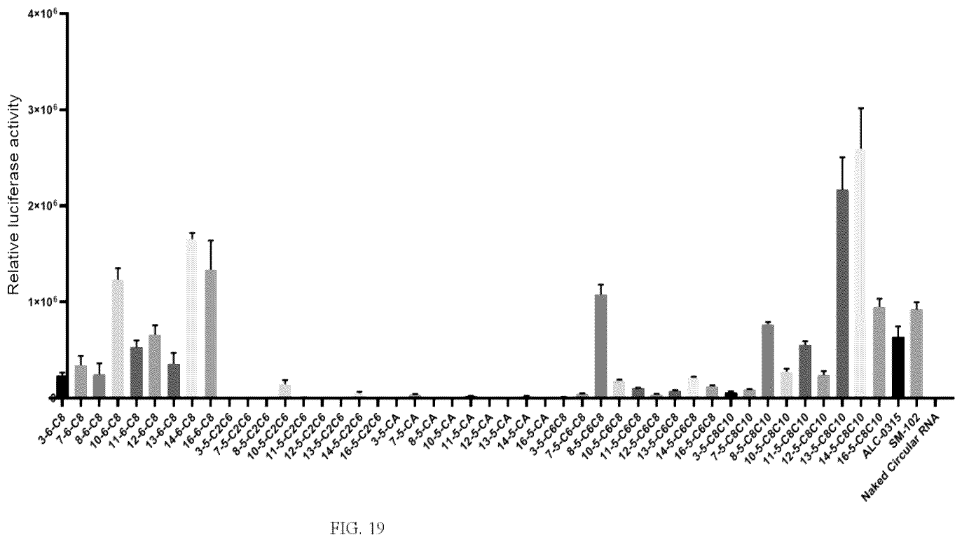

After 24 hours of cell transfection, the culture medium of the 96-well cell culture plate is completely sucked out. Cell lysis solution is added on the 96-well cell culture plate, and the cells are lysed on ice for 30 minutes. After centrifugation, the supernatant is extracted and transferred to a white 96-well detection plate. Firefly luciferase substrate is added on the white 96-well detection plate, and the firefly luciferase content is detected by utilizing an enzyme-linked immunosorbent assay (ELISA) reader (chemiluminescence). The relative luciferase activity results are shown in FIG. 19. The results show that the ionizable lipids synthesized by the present disclosure can enhance the transfection efficiency of circRNA. When tail of the ionizable lipid is 6-C8, 5-C6C8, and 5-C8C10, the RNA expression efficiency is highest. When the tail is 5-C2C6, 5-CA, the RNA expression efficiency is weaker. The transfection efficiency of lipid represented by 10-6-C8, 14-6-C8, 16-6-C8, 7-5-C6C8, 13-5-C8C10, and 14-5-C8C10 is superior to that of the commercial lipids ALC-0315 and SM-102, with transfection efficiency increased by 2˜3 times. This indicates that the ionizable lipids involved in the present disclosure have a wide range of applications and can be used for the delivery of different types of mRNA.

### Embodiment 21

LNP containing ionizable lipids 8-5-C8C10, 10-5-C8C10 or commercial lipids SM-102, ALC-0315 is used to deliver circRNA-Luc in Balb/c mice, and results are detected by utilizing the IVIS at 6 hours, 12 hours, 1 day, 2 days, 3 days, 4 days, and 5 days after intramuscular injection.
1. Specific steps refer to Embodiment 14 and Embodiment 19, the difference is that in Embodiment 21, the ionizable lipids used in the experimental group are 8-5-C8C10 and 10-5-C8C10. Ionizable lipids with a concentration of 10 mg/mL, DSPC with a concentration of 6 mg/mL, Cholesterol with a concentration of 12 mg/mL, and DSPE-PEG with a concentration of 5 mg/ml are dissolved in anhydrous ethanol in sequence. An appropriate amount of circRNA-Luc is adsorbed and dissolved in a sodium acetate buffer (volume of the sodium acetate buffer is twice total volume of the lipid mixture, and pH-5.3). The buffer containing circRNA-Luc is adsorbed and added into the lipid mixture solution, and quickly mixed evenly to assemble to be the LNP. The mixed solution is incubated at room temperature for 15 minutes, dialyzed in PBS for 1 hour by utilizing a dialysis bag (MWCO=14000 MW), and then performed for an intramuscular injection (LNP with 1.5 μg of circRNA-Luc injected per injection). The usage ratio is ionizable lipids: 8-5-C8C10 or 10-5-C8C10: Cholesterol: DSPC: DSPE-PEG=40:48:10:2. The solution of premixed RNA is sodium acetate, and the nitrogen phosphorus ratio of ionizable lipids and RNA is 18:1.

Positive control group: commercial lipids ALC-0315, SM-102, which are assembled LNP according to the disclosed preparation method. The specific operation is as follows. ALC-0315 or SM-102 with a concentration of 5 mg/mL, DSPC with a concentration of 1.5 mg/mL, Cholesterol with a concentration of 3 mg/mL, and ALC-0159 or DMG-PEG2000 with a concentration of 1 mg/mL are dissolved in anhydrous ethanol in sequence. They are mixed evenly in a molar ratio of ALC-0315: Cholesterol: DSPC: ALC-0159=46.3:42.7:9.4:1.6 or in a molar ratio of SM-102: Cholesterol: DSPC: DMG-PEG2000=50:38:10:1.5. At the same time, an appropriate amount of circRNA-Luc is adsorbed and dissolved in a sodium citrate buffer (volume of the sodium citrate buffer is three times total volume of the lipid mixture, and pH-4.0). Then the buffer containing circRNA and the lipid mixture solution are mixed evenly, and incubated at room temperature for 15 minutes to assemble to be a stable LNP. After dialysis in PBS for 1 hour by utilizing a dialysis bag (MWCO=14000 MW), intramuscular injection is performed (LNP with 1.5 μg of circRNA-Luc injected per injection). The nitrogen to phosphorus ratio between ALC-0315 or SM-102, and RNA is 6:1.
2. Analysis of In Vivo Imaging Results

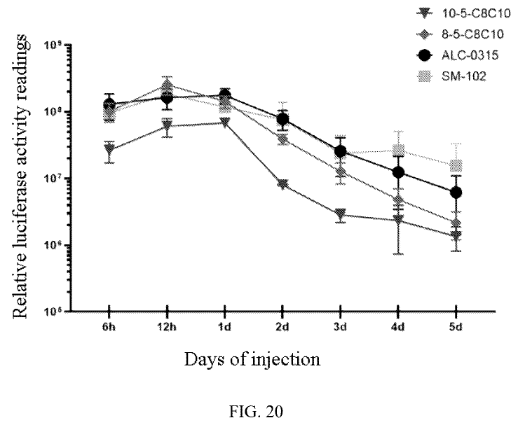

The IVIS results (FIG. 20) show that the expression peak of LNP loaded with circRNA-Luc is between 12˜24 hours. The expression intensity of ionizable lipid 8-5-C8C10 of the present disclosure is comparable to that of commercial lipids SM-102 and ALC-0315.

The above embodiments are preferred embodiments of the present disclosure, but the embodiments of the present disclosure are not limited by the above embodiments. Any other changes, modifications, substitutions, combinations, simplifications made without departing from the spirit and principles of the present disclosure should be equivalent substitution methods and are included in the scope of protection of the present disclosure.

What is claimed is:

1. A multi-tail type ionizable lipid, with a structural formula is as follows: 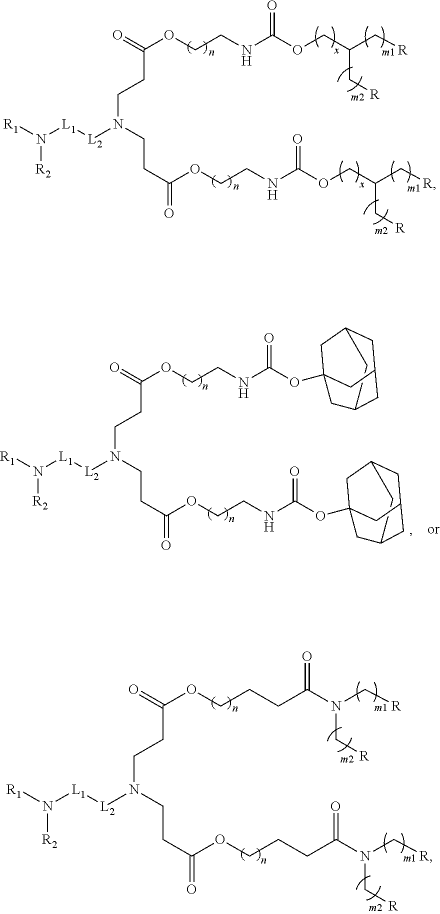
wherein, R₁ and R₂ are same or different, each consisting of hydrogen or an alkyl chain or an alkyl ring consisting of 1˜6 carbons, or R₁ and R₂ together form a nitrogen-containing alkyl ring; L₁ and L₂ are same or different, each consisting of an alkyl chain or an unsaturated hydrocarbon group with a length of 1˜6 carbons; R is an alkyl, an alkyl ring, an unsaturated alkyl or a heteroalkylene; n=1˜6; m1-1˜15, m2=1˜15; and x=0˜5.

2. The multi-tail type ionizable lipid according to claim 1, wherein the structural formula is selected from the following formulas:

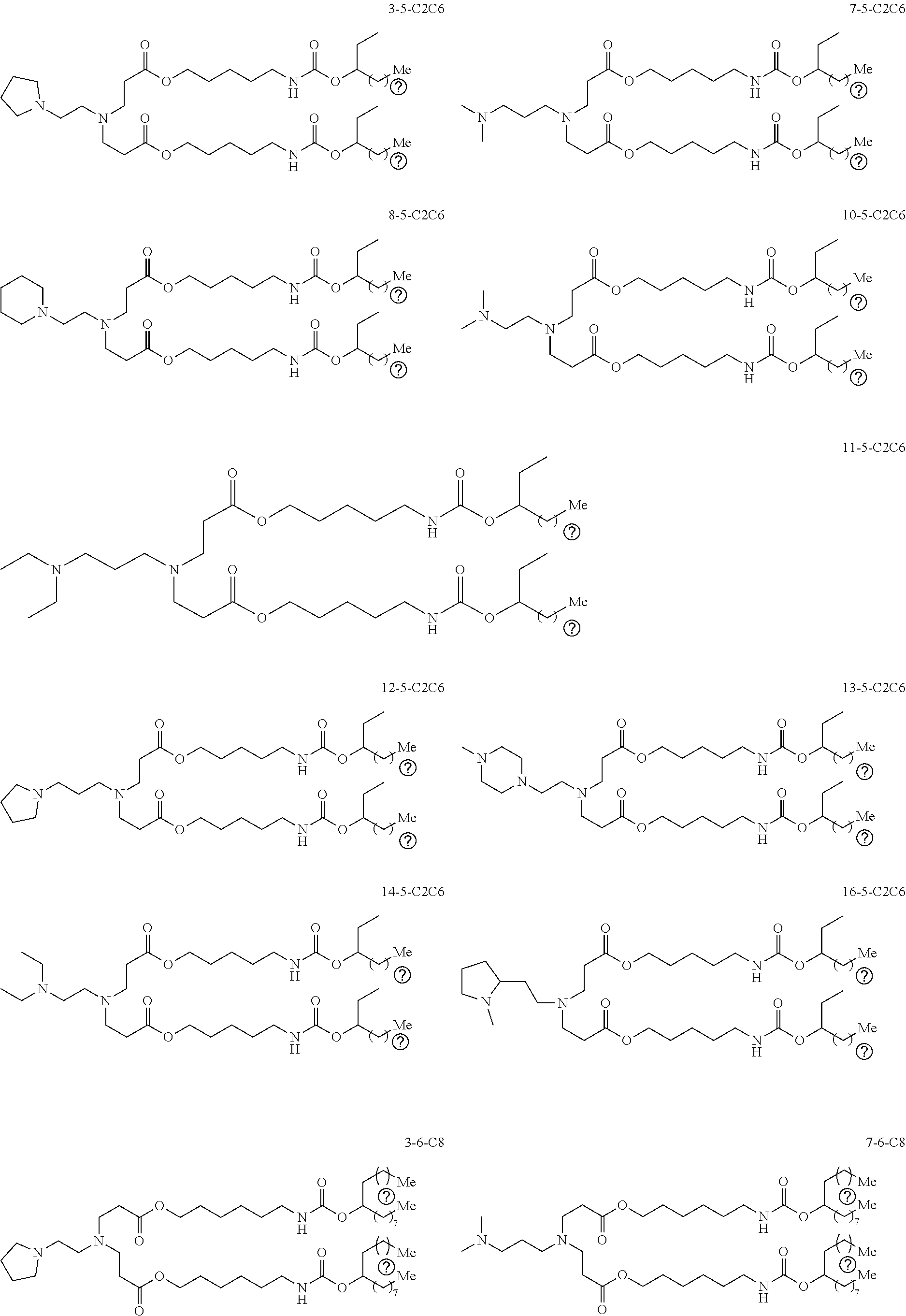

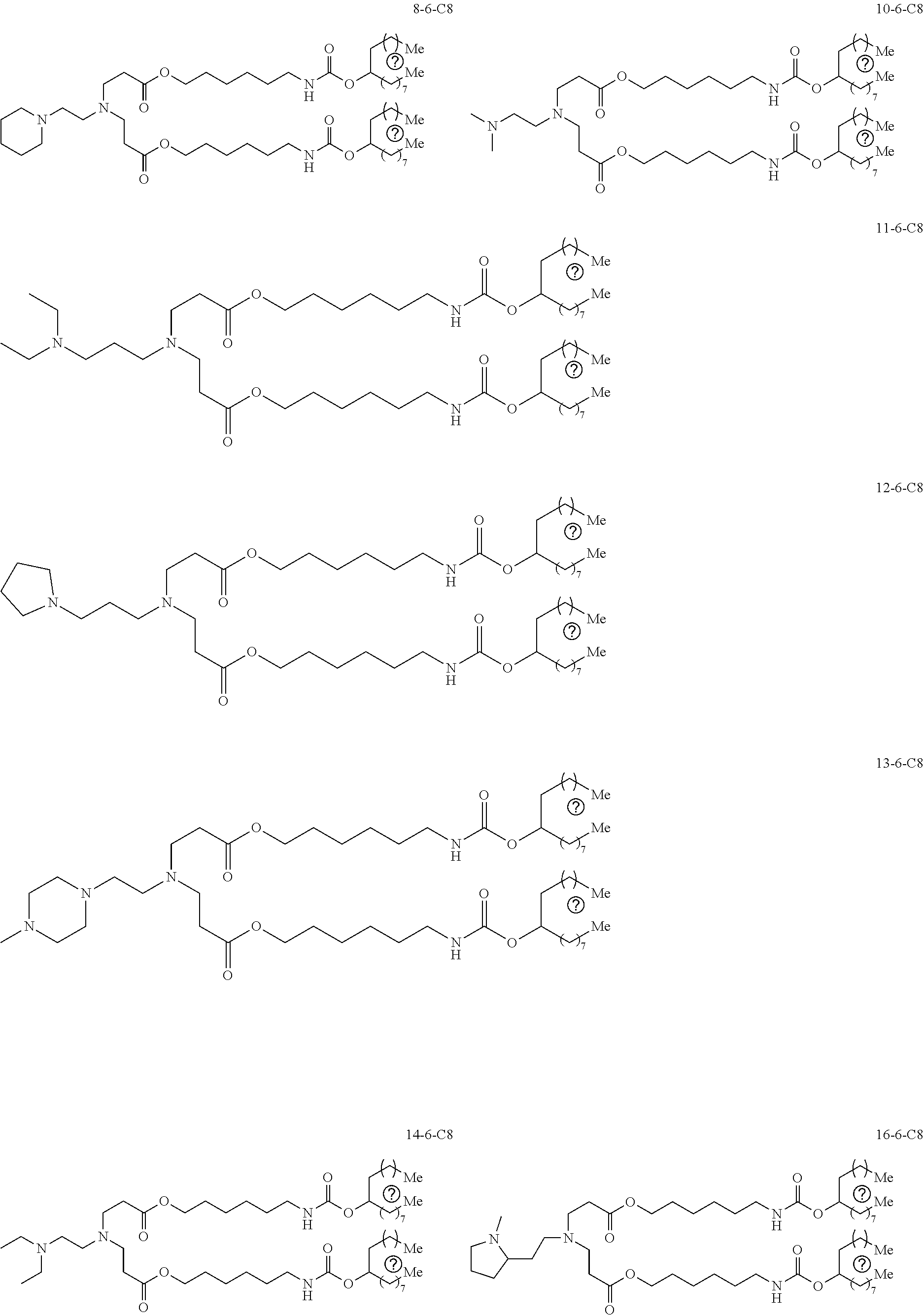

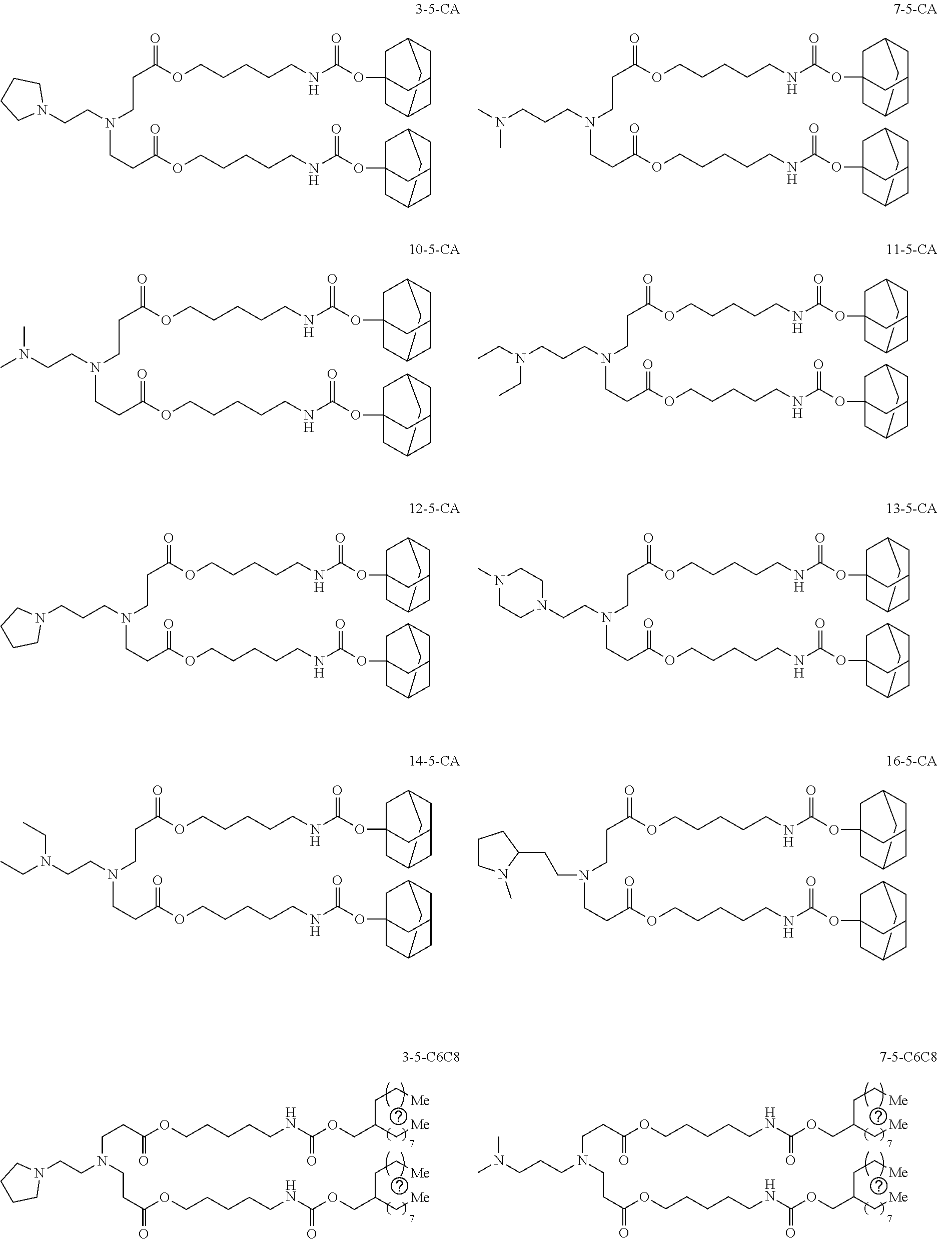

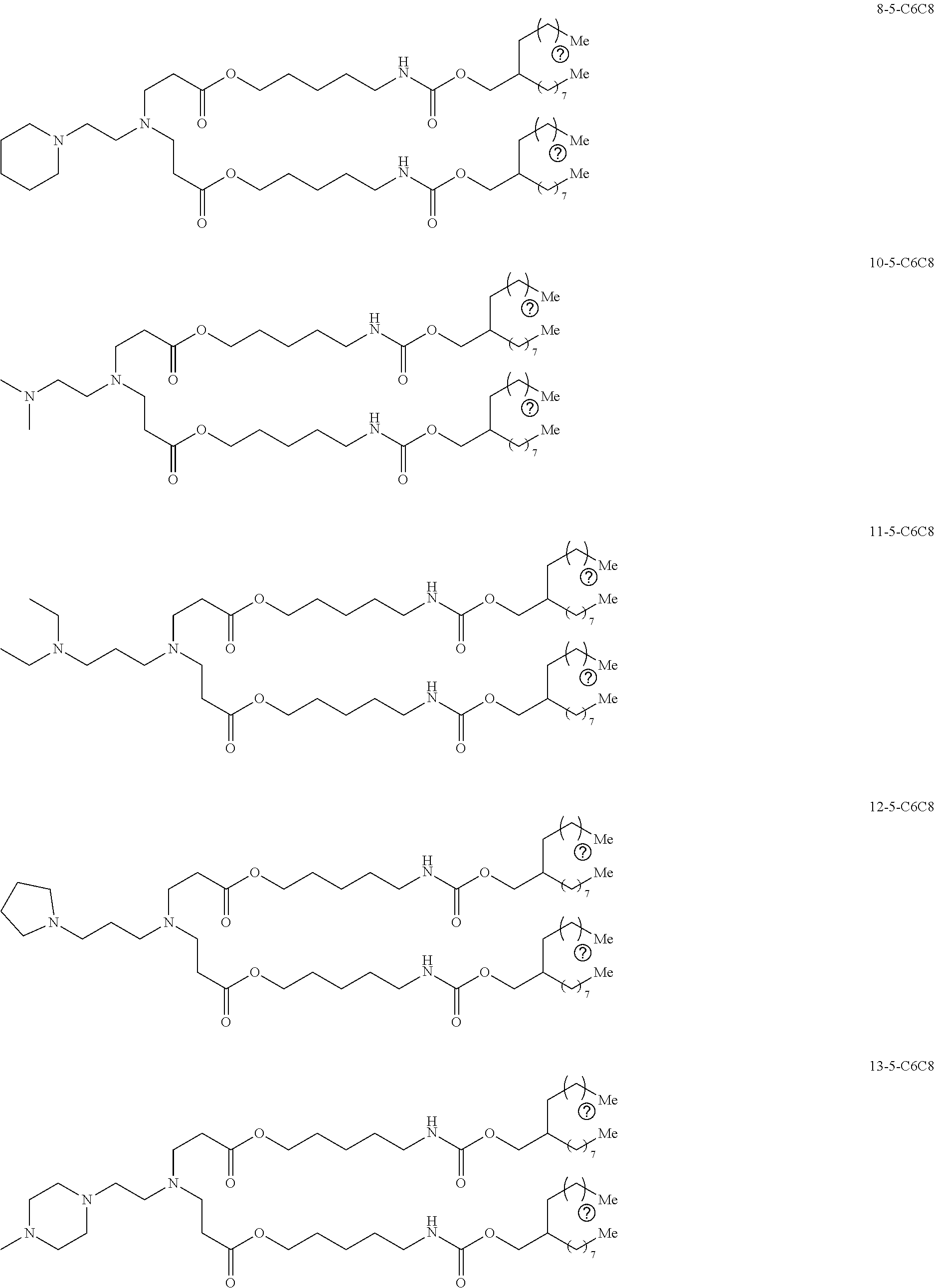

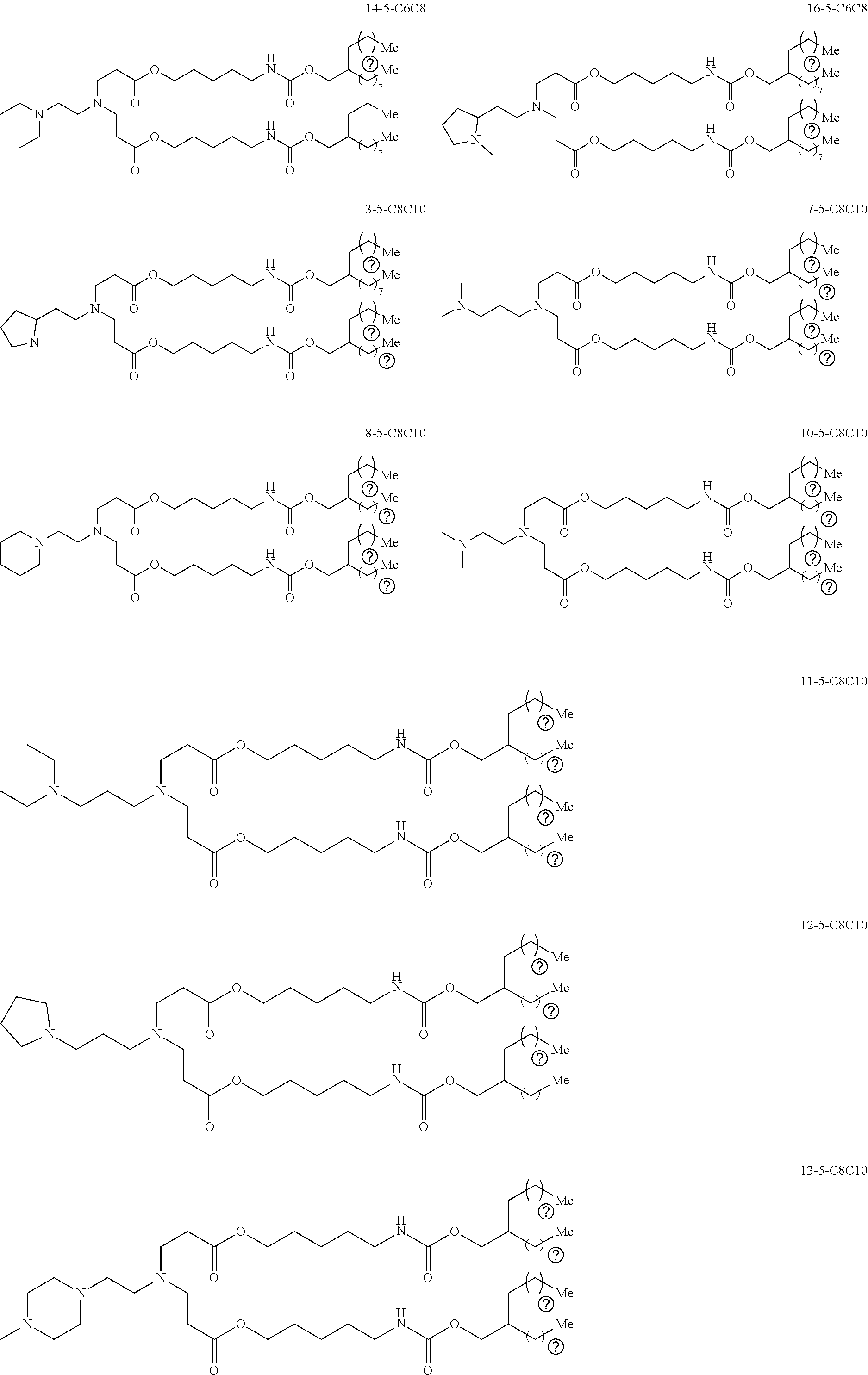

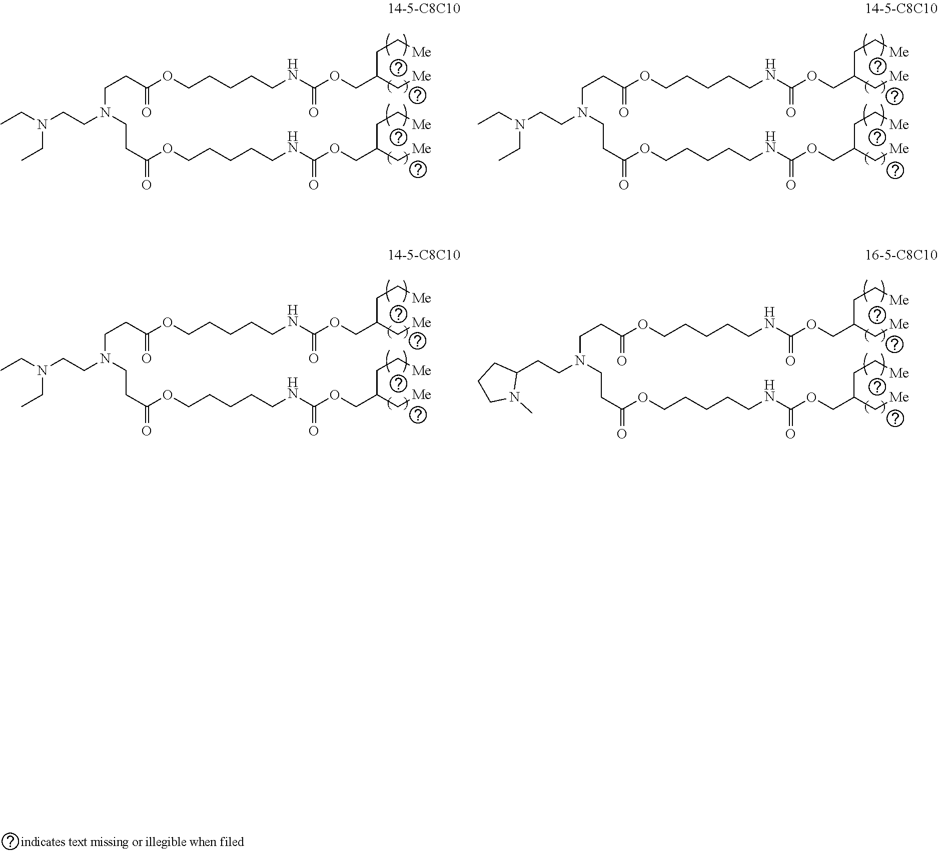

3. A method of preparing the multi-tail type ionizable lipid according to claim 1, wherein the multi-tail type ionizable lipid is obtained by performing a Michael addition reaction on organic amine compounds and tail compounds containing branched chains:
wherein a structure of the tail compound containing branched chains is as follows: 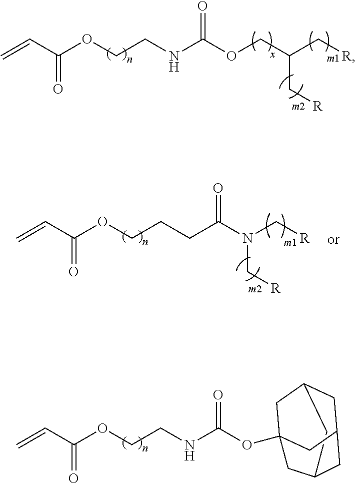
wherein, R is an alkyl, an alkyl ring, an unsaturated alkyl or a heteroalkylene; n=1˜6; m1=1˜15, m2=1˜15; and x-0˜5;
and wherein the organic amine compounds contain at least one amino group.

4. The method according to claim 3, wherein the organic amine compounds are selected from one of following compounds:

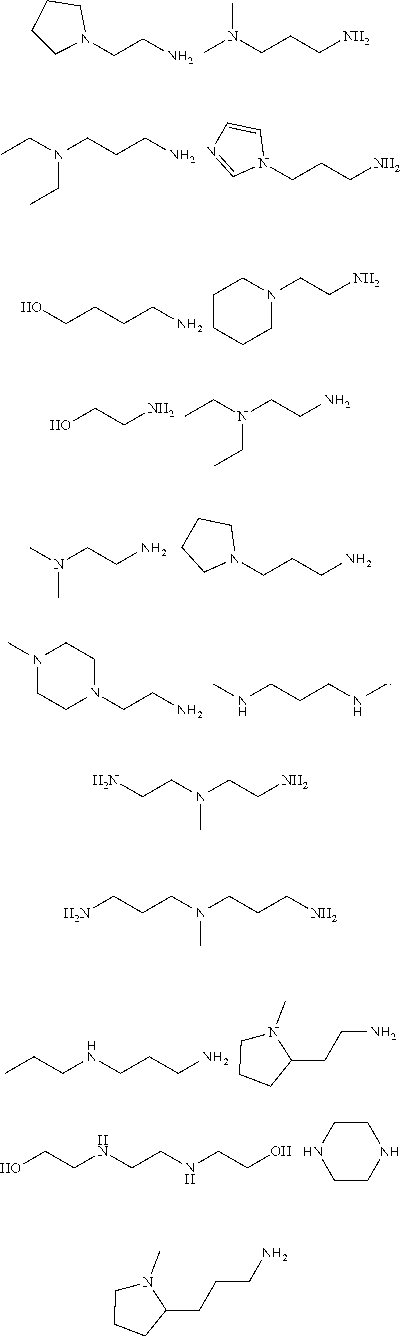

5. The method according to claim 3, wherein the tail compounds containing branched chains are obtained by esterification of chlorine acrylate with a compound having a structure as follows: 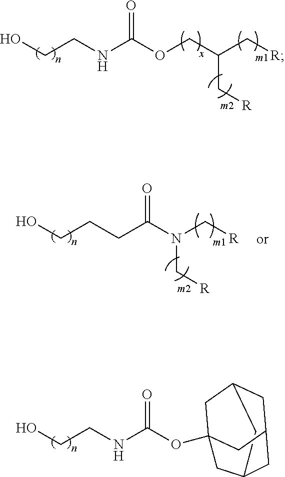
wherein, R is an alkyl, an alkyl ring, an unsaturated alkyl or a heteroalkylene; n=1˜6; m1=1˜15, m2-1˜15; and x=0˜5.

6. A method of preparing the multi-tail type ionizable lipid according to claim 2, wherein the multi-tail type ionizable lipid is obtained by performing a Michael addition reaction on organic amine compounds and tail compounds containing branched chains:
wherein a structure of the tail compounds containing branched chains is as follows: 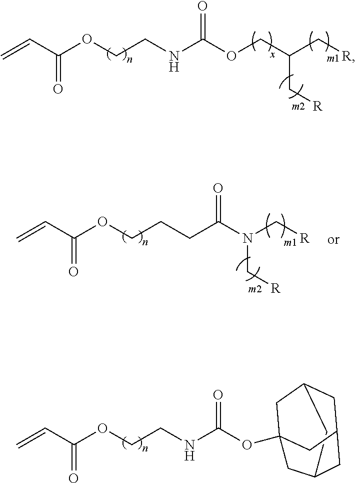
wherein, R is an alkyl, an alkyl ring, an unsaturated alkyl or a heteroalkylene; n=1˜6; m1=1˜15, m2=1˜15; and x-0˜5;
wherein the organic amine compounds contain at least one amino group.

7. The method according to claim 6, wherein the organic amine compounds are selected from one of following compounds:

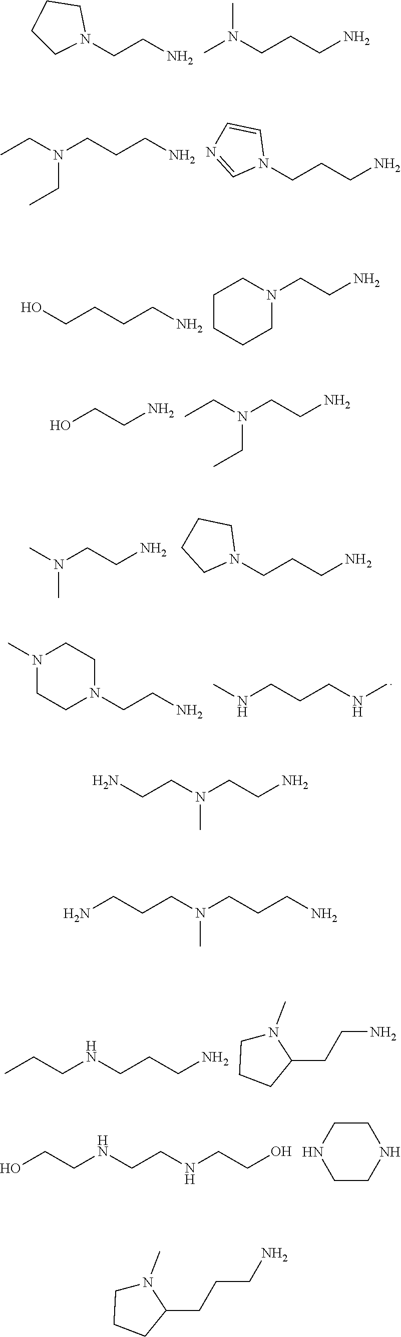

8. The method according to claim 6, wherein the tail compounds containing branched chains are obtained by esterification of chlorine acrylate with a compound 1;
and wherein a structure of the compound 1 is as follows: 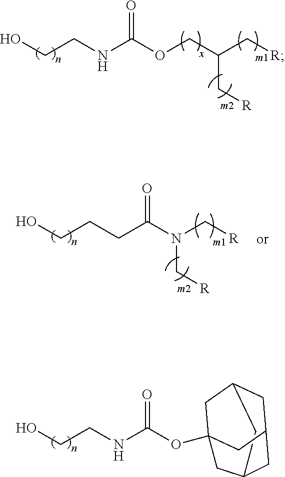
wherein, R is an alkyl, an alkyl ring, an unsaturated alkyl or a heteroalkylene; n=1˜6; m1=1˜15, m2=1˜15; and x=0˜5.

9. A method of use of multi-tail type ionizable lipid according to claim 1, comprising a step of formulating a drug wherein the multi-tail type ionizable lipid is used in preparation of a drug carrier.

10. The method according to claim 9, wherein active ingredients of the drug comprise nucleic acid molecules and protein drugs.

11. The method according to claim 10, wherein the nucleic acid molecules comprise siRNA, miRNA, mRNA, circRNA, antisense RNA, CRISPR guide RNAs, replicable RNA, cyclic dinucleotides, poly IC, CpG ODN, plasmid DNA, or micro circular DNA; and the protein drugs comprise cell colony-stimulating factors, interleukins, lymphotoxins, interferon proteins, tumor necrosis factors, antibodies, or protein antigens.

12. The method according to claim 9, wherein a preparation method of the drug carrier comprises following steps:
(a) mixing multi-tail type ionizable lipid with cholesterol or cholesterol derivatives, auxiliary lipids, and polyethylene glycol modified lipids in ethanol solution to prepare a lipid mixture solution; mixing a drug with an acidic buffer, and then mixing evenly with the lipid mixture solution; incubating at room temperature for 15 minutes to 1 hour, diluting with PBS or dialyzing to obtain the drug carrier;
or (b) dissolving the multi-tail type ionizable lipid with cholesterol or cholesterol derivatives in chloroform, drying with nitrogen to evaporate a solvent, adding acidic or neutral buffer and sonicating for 1˜20 minutes to prepare liposome nanoparticles for later use; mixing fish sperm protein with the drug, then mixing with the liposome nanoparticles, standing for 5˜30 minutes, adding polyethylene glycol modified lipids, and standing at 30˜65° C. for 5˜20 minutes to obtain the drug carrier.

13. The method according to claim 12, wherein in step (a), a ratio of an amount of substance of the multi-tail type ionizable lipid to cholesterol or cholesterol derivatives, auxiliary lipids, and polyethylene glycol modified lipids is 10˜100: 0˜90: 0˜90: 0˜90; a nitrogen to phosphorus ratio of protonated amino groups in the multi-tail type ionizable lipid to nucleic acid drug is 1˜100:1;
in step (a), the auxiliary lipids comprise at least one of egg yolk phospholipids, hydrogenated egg yolk phospholipids, soy phospholipids, hydrogenated soy phospholipids, sphingophospholipids, phosphatidylethanolamine, dimyristoylphosphatidylcholine, dimyristoylphosphatidylglycerol dipalmitoylphosphatidylcholine, distearoyl phosphatidylcholine, dioleoylphosphatidylethanolamine, dioleoylphosphatidylcholine, dioleoylphosphatidylcholine, and succinylphosphatidylcholine;
in step (b), a ratio of an amount of substance of the multi-tail type ionizable lipid to cholesterol or cholesterol derivatives is 1: 5˜5:1; a mass ratio of the multi-tail type ionizable lipid to the drug is 1˜100:1;
in steps (a) and (b), the polyethylene glycol modified lipids comprise at least one of DSPE-PEG, C14-PEG, DMG-PEG, ALC-0159, DSPE-PEG-Maleimide, DSPE-PEG-COOH, DSPE-PEG-NH₂;
in step (a), the acidic buffer has a pH of 3˜7; and the acidic buffer is a sodium acetate buffer or a sodium citrate buffer;
in step (b), the acidic buffer or the neutral buffer has a pH of 3˜7; the acidic buffer or the neutral buffer is a sodium citrate buffer, a sodium acetate buffer, or a DPEC water.

14. A drug carrier comprising a multi-tail type ionizable lipid according to claim 2.

15. The drug carrier according to claim 14, wherein active ingredients of a drug comprise nucleic acid molecules and protein drugs.

16. The drug carrier according to claim 15, wherein the nucleic acid molecules comprise siRNA, miRNA, mRNA, circRNA, antisense RNA, CRISPR guide RNAs, replicable RNA, cyclic dinucleotides, poly IC, CpG ODN, plasmid DNA, and micro circular DNA; and the protein drugs comprise cell colony-stimulating factors, interleukins, lymphotoxins, interferon proteins, tumor necrosis factors, antibodies, and protein antigens.

17. The drug carrier according to claim 14, wherein a preparation method of the drug carrier comprises following steps:
(a) mixing multi-tail type ionizable lipid with cholesterol or cholesterol derivatives, auxiliary lipids, and polyethylene glycol modified lipids in ethanol solution to prepare a lipid mixture solution; mixing a drug with an acidic buffer, and then mixing with the lipid mixture solution; incubating at room temperature for 15 minutes to 1 hour, diluting with PBS or dialyzing to obtain the drug carrier;
or (b) dissolving the multi-tail type ionizable lipid with cholesterol or cholesterol derivatives in chloroform, drying with nitrogen to evaporate a solvent, adding acidic or neutral buffer and sonicating for 1˜20 minutes to prepare liposome nanoparticles for later use; mixing fish sperm protein with the drug, then mixing with the liposome nanoparticles, standing for 5˜30 minutes, adding polyethylene glycol modified lipids, and standing at 30˜65° C. for 5˜20 minutes to obtain the drug carrier.

18. The drug carrier according to claim 17, wherein in step (a), a ratio of an amount of substance of the multi-tail type ionizable lipid to cholesterol or cholesterol derivatives, auxiliary lipids, and polyethylene glycol modified lipids is 10˜100: 0˜90: 0˜90: 0˜90; a nitrogen to phosphorus ratio of protonated amino groups in the multi-tail type ionizable lipid to nucleic acid drug is 1˜100:1;
wherein, in step (a), the auxiliary lipids comprise at least one of egg yolk phospholipids, hydrogenated egg yolk phospholipids, soy phospholipids, hydrogenated soy phospholipids, sphingophospholipids, phosphatidylethanolamine, dimyristoylphosphatidylcholine, dimyristoylphosphatidylglycerol dipalmitoylphosphatidylcholine, distearoyl phosphatidylcholine, dioleoylphosphatidylethanolamine, dioleoylphosphatidylcholine, dioleoylphosphatidylcholine, and succinylphosphatidylcholine;
wherein, in step (b), a ratio of an amount of substance of the multi-tail type ionizable lipid to cholesterol or cholesterol derivatives is 1:5˜5:1; a mass ratio of the multi-tail type ionizable lipid to the drug is 1˜100:1;
wherein, in steps (a) and (b), the polyethylene glycol modified lipids comprise at least one of DSPE-PEG, C14-PEG, DMG-PEG, ALC-0159, DSPE-PEG-Maleimide, DSPE-PEG-COOH, DSPE-PEG-NH₂;
wherein, in step (a), the acidic buffer has a pH of 3˜7; and the acidic buffer is a sodium acetate buffer or a sodium citrate buffer;
wherein, in step (b), the acidic buffer or the neutral buffer has a pH of 3˜7; the acidic buffer or the neutral buffer is a sodium citrate buffer, a sodium acetate buffer, or a DPEC water.
